(19) United States
(12) Patent Application Publication
(10) Pub. No.: US 2026/0226389 A1
(43) Pub. Date: Aug. 6, 2026

(54) FOAM DESTRUCTION DEVICE AND A PROCESS FOR FOAM DESTRUCTION

(71) Applicant: Boehringer Ingelheim International GmbH, Ingelheim am Rhein (DE)

(72) Inventors: Thomas WUCHERPFENNIG, Ulm (DE); Maike KUSCHEL, Ulm (DE); Juergen FITSCHEN, Hamburg (DE); Michael SCHLUETER, Hamburg (DE)

(21) Appl. No.: 19/150,203

(22) PCT Filed: Jan. 24, 2024
(86) PCT No.: PCT/EP2024/051598

§ 371 Date: Jul. 23, 2025

(30) Foreign Application Priority Data
Jan. 25, 2023 (EP) 23153210.2

(51) Int. Cl. C12M 1/21; C12M 1/00; C12M 1/06; C12M 1/12

(52) U.S. Cl. CPC C12M 41/02; C12M 23/06; C12M 23/26; C12M 27/02; C12M 29/06

(57) ABSTRACT

The present disclosure is directed to a foam destroying device for destroying foam (170) and/or controlling the foam height HF in a reactor (100) in which a foam-generating method is carried out, wherein a flexible tube (10, 180) attached to a rigid fluid line (185) only at one end (20, 181) which can come into contact with the liquid level (155) in a reactor (100) is caused to move chaotically by compressed fluid flowing there through, whereby the chaotic movement of the tube (10, 180) and the compressed fluid flowing out of the tube (10, 180) destroys or controls the foam (170) formed. A process for destroying foam (170) and/or controlling the foam height HF in a reactor (100) in which a foam-generating method is performed is also provided. Antifoam agents or mechanically-electronically operated or electronically driven components, such as motors, for destroying and suppressing foam are no longer required. The present disclosure is further directed to methods for culturing prokaryotic or eukaryotic cells in liquid cell culture in a bioreactor equipped with a respective foam destroying device and respective methods for producing a recombinant protein.

### TECHNICAL FIELD

The invention relates to a foam destruction device and a process for foam destruction.

### BACKGROUND OF THE INVENTION

It is well known that foam formation occurs in a large number of processes during the execution of a process. The foam is generated during the production or formation of a product, for example, by the starting materials, the stirrer or a gas sparging system used. The processes are, for example, chemical processes in which foam is produced during a reaction. They can also be biological or biopharmaceutical processes, which include, for example, the cultivation of organisms, such as cells or microorganisms, in suspension. Foam formation is usually continuous but uneven, and the foam can be characterized by the height of the foam layer formed or by the increase in the foam forming velocity. Foam formation is therefore a dynamic process. The thickness of the foam layer also depends on the properties of the liquid phase on which it is formed.

The formation of foam often has a negative effect on the product to be formed and the process to be carried out. Foam is an undesirable by-product that contaminates the product. For example, it is known that foam formation leads to problems in the cultivation of cells or microorganisms. Foam formation also has a negative effect on process control. It is particularly disadvantageous when the medium present in the reactor continues to foam up and eventually even begins to leak out of a reactor, whereby components of the reactor are impaired or even clogged by the foam, such as necessary ventilation or exhaust air filters. This can even lead to the termination of the process.

One way to remedy this is to use chemical additives for defoaming. For example, chemical additives in the form of anti-foaming agents, such as simethicone, can be added to the process to suppress foam formation. However, these antifoaming agents have numerous disadvantages: They have an undesirable effect on the product and can even change it to some extent. For example, it is well known that antifoams influence the growth of microorganisms and the metabolites produced, so that the product quality suffers. For instance, [1] describes that the addition of antifoaming agents can generally lead to major problems during cultivation. In addition, the cultivation process is even negatively affected in some cases, in particular the mass transfer is worsened and the oxygen transfer rate or oxygen saturation is significantly reduced. According to [2], in high density mass culture of rotifer, in which a large amount of foam is regularly produced, an increase of the viscosity of culture water and a reduction of the oxygen exchange rate between air and water occur with the addition of a defoamer. An excess of defoamer decreases the rotifer population.

In addition, antifoaming agents, such as simethicone, can also interfere with product separation and cleaning. In cleaning processes, these additives interfere as they can form micelles that are difficult to remove during cleaning. These chemicals are therefore usually considered process-related contaminants that need to be strictly controlled and minimised during development and manufacturing. The control and testing of antifoaming agents is a complicated, laborious and costly endeavour in process optimisation and commercialisation, especially as new processes are designed for higher yields, such as higher cell densities in cultivation processes, resulting in a greater propensity for heavy foaming and therefore requiring even more antifoaming agent.

In addition to chemical additives, mechanical means driven by motor can also be used to destroy foam. For example, mechanical foam-breakers can be used to destroy the foam. For example, [3] describes a mechanical foam-breaking rotating disk (MFRD) comprising a foam impact plate firmly attached to baffle plates on the walls of a bioreactor and having a rotating disk of the same thickness and size as the foam impact plate, installed in a stirred draft-tube bioreactor to provide effective foam-breaking action.

Furthermore, [4] and [4a] disclose an antifoaming device which comprises an impeller 61 that can be rotated (e.g., magnetically) using a motor 62, which may be external to the container. The mechanical foam breaker can also include one or more stainless steel plates or cones mounted on a hollow rotating shaft that penetrates the container. The shaft can be rotated by an external motor (e.g. a magnetically-operated motor) or other suitable apparatus.

Furthermore, [5] describes a fermentation defoaming device, comprising a fermentation tank main stirring shaft placed in a fermentation tank, paddle rods are arranged on both sides of the fermentation tank main stirring shaft, and a serpentine defoaming tube is arranged on the paddle rod; the two free ends of the paddle rod are provided with soft sleeves. When in use, the main stirring shaft of the fermentation tank rotates, and the paddle shaft rotates accordingly. The serpentine defoaming tube on the paddle shaft will repeatedly collide, agitate the bubbles on the liquid surface, continuously destroy the generated bubbles, and control the increase of foam.

Literature [6] discloses an antifoaming system such as a mechanical antifoaming device provided in a bag support structure 104 for a flexible bag in a mixing system. The antifoaming device is a second agitator 143 that is rotated using a second drive system 123 including a motor that is external to the flexible bag 128.

Document [7] refers to a very complicated automatic fermentation system defoaming device, in which a defoaming comb-tooth structure with a plurality of steel needles are arranged side by side under a main board, radially fixedly mounted on the upper portion of a stirring shaft in a fermenting tank. Furthermore, a plurality of pressure plates are fixedly disposed on an upper circumference of the agitating shaft 5, located above the defoaming comb 3. The foam is sucked out by the suction pump 9 through the suction pipe 7 above the pressure plates.

Moreover document [8] describes a reactor which is driven in an oscillating-rotating manner about a fixed vertical axis for biotechnological and pharmaceutical applications, the reactor is preferably a disposable reactor. The oscillatory-rotary motion of the reactor and the power input is set in such a manner that foam formation on the surface of the reactor content should be minimized.

However, the above systems based on mechanically driven motors to destroy the foam are often complex devices that are either complicated to build and set up or can only be used in plants that are agitated from above, as they are passively installed on the agitator shaft. This technique works only at high agitation speeds. In addition, the solutions that have their own motor must be sealed against the environment at great expense, and during cleaning (CIP process, cleaning in place or site-specific cleaning), the very complex set-up with complicated geometry must be cleaned at great expense in terms of time and money.

Such mechanical solutions driven by a motor having moving parts not only have the major disadvantage that they are expensive to design and manufacture, as they usually have to be adapted to the respective device, such as a bioreactor, but the replacement of broken parts is also difficult.

In the state of the art, there are also proposals in which mechanical means driven by motor are combined with chemical additives. This then leads to a combination of the adverse influences and effects already described.

Another disadvantage of existing solutions for active foam destruction is that they cannot be applied in a simple way to existing devices, for example reactors, but require a completely new design, especially a special reactor design ([1], [8]).

Another alternative to foam destruction is foam avoidance. One possibility in the cultivation of organisms is to reduce the gassing rate by switching to pure oxygen instead of air aeration. This reduces foaming, as the foaming rate is directly proportional to the gassing rate. This is described in [2], for example, where only one fifth of the volume is required for gassing with pure oxygen instead of air in a bioreactor. Less gas supply means less foam. An alternative solution approach, [9] uses an open tube gas sparger to create a larger number of very large bubbles, which contribute less to foam formation. However, both proposed prior art solutions have the disadvantage that they directly affect the mass transfer performance and thus actively interfere with and change the process, which is actually entirely undesirable. Therefore, these solutions are not applicable in practice for existing devices, especially reactors, and processes.

Furthermore, document [10] discloses a disposable bioreactor with foam destroyer, comprising: a flexible container bag for containing a liquid phase; an agitator for agitating the liquid phase, which is pivotally supported in the container bag; and at least one foam destroying device, which is designed to at least partially destroy mechanically and/or chemically foam which has formed on a surface of the liquid phase in the container bag. The foam-destroying device comprises at least one spray nozzle which is formed and/or mounted in a container wall of the container bag in such a way that a fluid can be supplied from the outside to an interior of the container bag and sprayed onto a foam which has formed on a surface of the liquid phase in the container bag in order to at least partially destroy the foam.

Document describes a container which has flexible walls surrounding a container interior. At least one electrical sensor is installed into the container interior and has at least one or two electrically conductive plates for determining the conductivity or impedance of a medium that surrounds the plates. The plates are connected via connecting lines to a control and regulating unit outside the container interior. The sensor formed from at least two plates may be designed for detection of fluid and/or foam.

Document is directed to systems for liquid level and foam monitoring and regulation in a vessel, such as a bioreactor. The systems use infrared devices, such as cameras and sensors, to detect foaming and automated control of defoaming is performed by using defoaming agents.

It is therefore an object of the present invention to overcome the deficiencies of prior art and to provide a foam destruction device which allows foam destruction or foam control in a method during which foam formation takes place, without the use of anti-foaming additives and dispensing with mechanical means driven by motor, particularly mechanically-electronically or electronically operated components to destroy the foam. The device shall be applicable from laboratory up to industrial scale. Furthermore, it is a further object to provide a process to destroy or control the foaming in a foam-producing method which allows foam destruction or foam control without the use of anti-foaming additives and dispensing with mechanical means driven by motor, particularly mechanically-electronically or electronically operated components to destroy the foam. The process should be feasible on a laboratory or industrial scale.

### SUMMARY OF THE INVENTION

Surprisingly, it was found that the disadvantages known from prior art can be overcome by using a tube for foam control or foam destruction, which, by passing compressed fluid through it, performs chaotic and thus uncontrollable movements and thereby at least partially destroys the foam.

The invention therefore relates to a foam destruction device for destroying foam and/or controlling the foam height H_{F} in a reactor in which a foam-generating method is carried out, comprising:
a reactor which is to be filled or is filled with a liquid medium having a liquid level;
a flexible tube having a first end, a second end, a selected length L_{T} and a selected inner and outer diameter;
a rigid fluid line located above the liquid level of the liquid medium to be filled into the reactor or with which the reactor is filled;
a fluid connection that is connected to the rigid fluid line;
   whereby
the first end of the tube is attached to the fluid line to allow compressed fluid to pass through the tube;
the second end of the tube is not fixed but is free to move so as to be able to create a chaotic pattern of movement of the second end of the tube by the compressed fluid flowing through the tube; and
the length L_{T} of the tube is chosen such that the second end of the tube comes into contact with the liquid level of the liquid medium to be filled into the reactor or with which the reactor is filled, for the case of the maximum filling volume. Alternatively, the length L_{T} of the tube is chosen such that the second end of the tube does not come into contact with the liquid level of the liquid medium to be filled into the reactor or with which the reactor is filled.

Also a subject-matter of the present invention is to provide a process for destroying foam and/or controlling the foam height H_{F} in a reactor in which a foam-generating method is to be carried out, comprising the steps of:
providing a reactor in which a method is to be carried out in which foaming occurs comprising a liquid medium having a liquid level;
providing a flexible tube having a first end, a second end, a selected length L_{T} and a selected inner and outer diameter,
providing a rigid fluid line that is placed above the liquid level of the liquid medium in the reactor;
providing a fluid connection that is connected to the rigid fluid line;
attaching a first end of the tube to the fluid line connected to the gas connection, the second end of the tube not being fixed and remaining free to move;
allowing the tube to hang loosely downwards from the point of attachment with the fluid line into the reactor, the length L_{T} of the tube being chosen such that the second end of the tube comes into contact with the liquid level of the liquid medium in the reactor, for the case of the maximum filling volume, or the length L_{T} of the tube being chosen such that the second end of the tube does not come into contact with the liquid level of the liquid medium;
as soon as foam forms in the foam-generating method, adjusting the fluid flow through the tube so that the second end of the tube performs a chaotic pattern of movement in the reactor; and
destroying the formed foam and/or controlling the foam height H_{F} of the formed foam by contact of the tube with the foam and by the compressed fluid exiting from the tube.

The invention is also directed to a method (foam-generating method) for culturing prokaryotic or eukaryotic cells in liquid cell culture in a bioreactor, wherein foam is generated during the method, wherein a foam destroying device according to the present invention is used in the bioreactor performing a process for destroying foam and/or controlling the foam height H_{F} in the reactor according to the present invention.

It is also disclosed a method (foam-generating method) for producing a recombinant protein, the method comprising the steps of:
step a) culturing prokaryotic or eukaryotic cells expressing a recombinant protein in cell culture in a bioreactor, wherein foam is generated during culturing of the cells;
step b) harvesting the recombinant protein;
step c) purifying the recombinant protein;
wherein in step (a) a foam destroying device according to the present invention is used in the bioreactor performing a process for destroying foam and/or controlling foam height H_{F} in the reactor according to the present invention.

The invention is therefore based on the concept of active foam destruction or control, in which a flexible tube fastened to a fluid line at only one end above the liquid level in a reactor and by compressed fluid flowing through it is set in chaotic motion, whereby the chaotic motion of the tube and the compressed fluid flowing out of the tube destroys or controls the foam formed.

According to the invention, it is thus possible to destroy or control the foam without the addition of chemical additives, such as anti-foaming agents, or recourse to electronically or mechanically-electronically driven components.

### BRIEF DESCRIPTION OF THE DRAWINGS

Embodiments of the prior art and of the present invention will be described by way of example with reference to the accompanying figures, which are schematic and are not intended to be drawn to scale so that no assumption of precise geometric values can be made regarding the original size. The figures of the present disclosure are incorporated in and constitute a part of the specification, also illustrating embodiments of the invention without limitation to the specific embodiments described. The drawings together with the summary and detailed description serve to explain the principles of the present disclosure. The same features are denoted by the same reference signs throughout the figures. In the figures show:

FIG. 1a a schematic three-dimensional representation of an embodiment of a tube used according to the invention;

FIG. 1b a top view of the second end of the tube of FIG. 1a;

FIG. 2a, 2b, 2c schematically illustrate examples of 3 movement patterns of a tube in a reactor by means of a top view of the movement of the second free end of a tube;

FIGS. 3a and 3b simplified schematic sectional views of bioreactors with a device for foam destruction and/or control, each comprising one flexible tube according to an embodiment of the invention;

FIGS. 3c to 3f simplified schematic sectional views of reactors with a device for foam destruction and/or control, each comprising 2 flexible tubes according to an embodiment of the invention;

FIG. 4 the minimum gas flow rate Vm in [m³ s⁻¹] as a function of the tube length in [mm] with variation of the inner and/or outer diameter in [mm] of a tube, according to example 1;

FIG. 5 the dimensionless quotient of the momentum force F_{I} and weight force F_{G} against the tube length in [mm] when varying the inner and/or outer diameter in [mm] of a tube, according to example 2;

FIG. 6a.1 a photograph of a reactor during a foam-generating method illustrating the embodiment according to example 3 of the present invention;

FIG. 6a.2 a contour drawing of the photo of FIG. 6a.1;

FIG. 6b.1 an enlarged section of the photograph of FIG. 6a.1 showing foaming without active foam control (without the device of the invention) at a time during the foaming forming method;

FIG. 6b.2 a contour drawing of the photo of FIG. 6b.1;

FIG. 6c.1 an enlarged section of the photograph of FIG. 6a.1 showing foam formation with active foam control (with the device of the invention) according to an embodiment of the present invention at a time during the foam-generating method;

FIG. 6c.2 a contour drawing of the photo of FIG. 6c.1

FIG. 7a a large number of tests according to example 4 in which the foam level height H_{F} in [mm] is plotted against the sampling time in [s], with the copied-in photos illustrating the foam height H_{F} of individual samples;

FIG. 7b FIG. 7a wherein the photos have been replaced by contour drawings of the photos;

FIG. 7c FIG. 7a without the photos, to get a better impression of the curves formed by the measured values;

FIG. 8a an experiment according to example 5 in which the foam formation in a reactor without foam control and with active foam control according to an embodiment of the present invention during a foam-forming process is depicted in photos over time;

FIG. 8b contour drawings of the photos of FIG. 8a;

FIG. 9a a photograph of an industrial scale reactor with a partially active foam control according to the embodiment of example 6 of the present invention;

FIG. 9b a contour drawing of the photo of FIG. 9a;

FIG. 10 experiments according to example 8 in which the foam level height H_{F} measured in [mm] plotted against the measurement time t in [s] in a single-use stirred tank reactor (SUB) with or without active foam control according to an embodiment of the present invention; and

FIGS. 11a and 11b illustrations to explain the foam height H_{F} according to FIG. 10.

The legends of the figures are provided at the end of the description.

### DETAILED DESCRIPTION OF THE INVENTION

### Definitions

Terms not specifically defined herein should be given the meanings that would be given to them by a person skilled in the art in light of the disclosure and the context.

The expression “process” stands for the process according to the invention in different embodiments by which foam is destroyed and/or the foam height H_{F} is controlled.

The expression “method” stands for a foam-generating method, which shall not be further limited. This may represent any method by which foam is generated. The method shall take place or is carried out in a reactor, the foam-generating method is the method in which foam generation is to be controlled.

The term “fluid” is understood here to mean a gas or a mixture of gases or a liquid or a mixture of liquids all of which have no adverse effect on the foam-generating method carried out in the reactor. The gas or gases are not further limited and may be any suitable gas such as air, oxygen, any type of inert gas such as nitrogen or a noble gas, in particular argon. The liquid or liquids are also not further limited und may be any suitable liquid such as water, organic solvent or the liquid medium already used in the reactor.

The term “compressed fluid” is understood here to mean a fluid which is put under pressure to exit the tube under pressure and set it in chaotic motion. Exemplarily mentioned compressed fluids are compressed air, compressed oxygen, compressed inert gas such as nitrogen, compressed noble gas, in particular argon, compressed water, compressed organic solvent or compressed liquid medium already used in the reactor.

A “reactor” is understood to be a vessel, container, receptacle, apparatus or device in which a method can be carried out under defined conditions and controlled accordingly, with foam being generated or formed during the method. A foam-generating method may be any procedure known to the skilled person. Foam-forming methods are, for example, those in which surface-active substances are used or the cultivation of certain microorganisms, especially fermentations, whereby undesirable foam can occur. For example, the reactor may be a chemical reactor, a bioreactor including a fermenter, or any other known type of reactor.

A “chemical reactor” is a vessel, container, receptacle, apparatus or device in which a chemical reaction occurs under specified conditions.

A “bioreactor,” also referred to herein as a “fermenter”, is a vessel, container, receptacle, apparatus or device in which living organisms, especially certain microorganisms, cells or small plants, are cultivated or fermented under the best possible conditions. A bioreactor may consist of or comprise a biocompatible vessel in which a chemical or biochemical method is carried out which involves organisms and/or biochemically active substances derived from such organisms. A bioreactor uses additional equipment, for example stirrers, baffles, one or more spargers and/or ports, which specifically allows for the cultivation and propagation of the cells. Commonly the bioreactor is in the form of a cylindrical tube, having two end parts, the end parts forming the top and the bottom of the bioreactor. The bioreactor ranges in size from litres to cubic metres and is often made of stainless steel designed for multiple use. The bioreactor may also be designed for single use (SUB). Cultivation in a bioreactor is used to obtain the cells or cell components or metabolic products. These are used, for example, as active ingredients in the pharmaceutical industry for the production of drugs, e.g. as antibiotics, antibodies or insulin; or as basic chemicals in the chemical industry, e.g. in wastewater treatment, in the food industry, in pest control or in the biological degradation of waste or pollutants, e.g. in oil spills. The bioreactor according to the present disclosure may be used from laboratory scale up to large-scale production.

The foam-generating method according to the invention may be a method of cell cultivation. The term “cell cultivation” or “cell culture” includes cell cultivation and fermentation methods in all scales (e.g. from micro titer plates to large-scale industrial bioreactors, i.e. from sub mL-scale to >10000 L scale), in all different method modes, e.g. batch, fed-batch, perfusion, continuous cultivation, in all method control modes (e.g. non-controlled, fully automated and controlled systems with control of e.g. pH, temperature, oxygen content), in all kind of fermentation systems (e.g. single-use systems, stainless steel systems, glass ware systems). In a preferred embodiment the cell culture is a cell culture in a volume of >1 L, preferably >2 L, >10 L, >1000 L, >5000 L and more preferably >10000 L.

The cells cultivated in a bioreactor, especially eukaryotic cells like chinese hamster ovary (CHO) or yeast cells are for example used to produce antibodies such as monoclonal antibodies and/or recombinant proteins such as recombinant proteins for therapeutic use. Alternatively the cells may produce, for example, peptides, amino acids, fatty acids or other useful biochemical intermediates or metabolites or any other useful substances.

The term “eukaryotic cell” as used herein refers to cells that have a nucleus within a nuclear envelope and include animal cells, human cells, plant cells and yeast cells. In the present invention an “eukaryotic cell” particularly encompasses mammalian cell, such as Chinese hamster ovary (CHO) cell or HEK293 cell derived cells, and yeast cells.

The term “recombinant protein” as used herein relates to a protein generated by recombinant techniques, such as molecular cloning. Such methods bring together genetic material from multiple sources or create sequences that do not naturally exist. A recombinant protein is typically based on a sequence from a different cell or organism or a different species from the recipient host cell used for production of the protein, e.g., a CHO cell or a HEK 293 cell, or is based on an artificial sequence, such as a fusion protein. In the context of the present invention the recombinant protein is preferably a therapeutic protein, such as an antibody, an antibody fragment, an antibody derived molecule (e.g., scFv, bi- or multi-specific antibodies) or a fusion protein (e.g., a Fc fusion protein).

The term “expressing a recombinant protein” as used herein refers to a cell comprising a DNA sequence coding for the recombinant protein, which is transcribed and translated into the protein sequence including post-translational modifications, i.e., resulting in the production of the recombinant protein in cell culture.

The term “cell culture medium” as used herein is a medium to culture cells, e.g. mammalian cells, comprising a minimum of essential nutrients and components such as vitamins, trace elements, salts, bulk salts, amino acids, lipids, carbohydrates in a preferably buffered medium. Typically a cell culture medium for mammalian cells has an about neutral pH, such as a pH of about 6.5 to about 7.5, preferably about 6.8 to about 7.3, more preferably about 7. Non limiting examples for such cell culture media include commercially available media like Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), CHO—S—Invitrogen), serum-free CHO Medium (Sigma), and protein-free CHO Medium (Sigma) etc. as well as proprietary media from various sources. The cell culture medium may be a basal cell culture medium. The cell culture medium may also be a basal cell culture medium to which the feed medium and/or additives have been added. The cell culture medium may also be referred to as fermentation broth.

As background information for the cultivation of cells, type of cells, media used therefore and methods, reference is made to WO 2021/165302 A1, the contents of which be incorporated by reference in their entirety in the present disclosure. In particular for the definition of terms, particularly fed-batch, recombinant virus production, cell culture medium, basal medium, basal cell culture medium, feed, feed medium, feed supplement, chemically defined medium, protein-free medium, mammalian cell, coding for, heterologous protein and the like, reference is made to paragraphs [0054] to [0088] of WO 2021/165302 A1.

The term “laboratory scale” refers to an experimental set-up in which work is carried out on a small scale. These are, for example, approaches in which a volume of several mL to several litres is used.

The expressions “industrial scale” or “large-scale” are used interchangeably and synonymously and relate to a product which is obtained in a large production amount whereby there is often a cost advantage with costs per unit of output decreasing with increasing scale. A large manufacturing unit is to be expected to have a lower cost per unit of output than a smaller unit, all other factors being equal. An industrial scale may be understood in connection with the cultivation of cells to have a volume of the bioreactor used which is equal or greater than about 100 L. According to a further embodiment the volume of the bioreactor used in industrial scale may be equal or greater than 600, 800, 1,000, 1,200, 1,500 L or even more.

According to the invention, a “flexible tube” is understood to be a line or hose suitable for carrying compressed fluid and having flexibility so that it can be used for foam destruction and/or control. The flexible tube is an elastic hose which changes its shape when a force is applied and returns to its original shape when the applied force is removed.

The expressions “comprising”, “comprise”, “comprised”, “containing”, “containing” or “contained” shall also encompass the more specific term “consisting of” unless otherwise stated or apparent from the context.

In addition, it should be noted that in this disclosure, the singular and plural forms are not used in a restrictive manner. As used herein, the singular forms “a”, “an”, “one” and “the” therefore refer to both the singular and the plural, unless otherwise stated or apparent from the context.

The expression “about” or “approximately” means within 10%, particularly within 5% and more particularly within 1% or within 0.1% of a value specified or an upper or lower range value as indicated.

### EMBODIMENTS OF THE INVENTION

In the following, embodiments of the invention are described in detail. The embodiments and explanations apply accordingly to both the device and the process of the present invention.

The invention relates to a foam destruction device and a process for destroying foam and/or controlling the foam height H_{F} in a reactor in which foam is formed during a method. The foam destruction device according to the invention and the process according to the invention are also referred to herein as an active foam destruction and/or foam control or active foam control (device or process).

A method in which foaming occurs can be any type of chemical, physical, biological, biochemical, in particular biotechnological or biopharmaceutical process in which foam is produced. This can be, for example, a chemical method in which a product is produced by chemical reaction of two or more starting materials, during which foaming results. It may also represent, for example, a cultivation method of organisms, in particular cells or microorganisms, in which foam formation is observed during the cultivation method.

Destroying the foam and/or controlling the foam height H_{F} means that the foam disappears or is reduced to a desired maximum foam height H_{F(max)} by the foam destruction device or foam destruction process and can be maintained approximately at this foam height during the foam-forming method.

The device or process of the invention comprises a reactor in which a foam-generating method is to take place. The reactor is not further limited according to the invention. Any type of reactor can be used. Exemplary reactors may be mentioned, in particular chemical reactors, bioreactors including fermenters, which may be designed for multiple use or single use. For the device according to the invention or the process according to the invention for foam destruction and/or foam control, therefore, any type of vessel or also a process-engineering plant can be used as a reactor. The device or process of the invention may be suitable for or carried out on a laboratory scale or industrial scale.

According to an embodiment of the invention, the reactor is a so-called disposable reactor or single use bioreactor (SUBs). Disposable bioreactors can be used to cultivate cells in large quantities. In contrast to conventional reusable system reactors, which are made in particular of stainless steel, the cell culture vessel in SUBs is usually a plastic bag that is disposed of after use. The culture bag is provided sterile and installed in a bag holder that provides structural support and heating capabilities. The culture bag contains pre-prepared stirring elements as well as pre-prepared gassing and feed lines and sensor connections for process monitoring and control. There are various configurations of disposable bioreactors, most of which are modular in design and can therefore be easily modified and adapted to meet specific requirements. SUBs can be used in a particularly advantageous manner for the device or process according to the invention. It is advantageous here that a SUB is always used only for the cultivation of one culture, so that the flexible tube or tubes for foam destruction and/or control are already present from the outset. This makes it even easier to use a tube for foam destruction. This is especially due to the fact that in SUBs the connections and tubes are already pre-installed. Therefore, no great effort is required to attach one flexible tube or another tube at a suitable position.

In the reactor a liquid medium will be or is present. The liquid medium is not further limited, and can be any type of liquid. It can be selected from water, one or more organic solvents or any solvent/water mixtures. In particular, when a cultivation method is to be carried out, water is present as the liquid medium. As soon as the reactor is filled with liquid medium, the liquid medium is present in the reactor in such a large quantity that a liquid level is formed. It is assumed that only in this case foam can be formed.

The liquid medium contains chemical, biochemical or biological components that are present in liquid, semi-solid or solid form, for example. These can be, for example, chemical compounds that react with each other in the liquid medium or biological units such as cells or microorganisms that are cultivated in suspension in the liquid medium.

The foam-generating method in the reactor is a method during which foam formation occurs. Foam is understood to be a dispersion of gas bubbles in the liquid present, whereby the foam is lighter than the liquid and can therefore be found on the liquid and accumulate there.

A flexible tube with a first end, a second end, a selected length L_{T} and a selected inner diameter and a selected outer diameter is used to control and/or destroy the foam formed in the reactor. The tube is selected so that it is flexible and in particular also mechanically stable. The term “flexible” should be understood to mean that the tube is elastic. The tube is to be understood as elastic in the classical sense, so that it changes its shape when a force is applied, for example bends, and returns to its original shape when the applied force is removed. The tube is therefore flexible, in particular easily bendable.

The term “mechanically stable” in this context means a resistance to tearing or breaking due to mechanical stress. During its use, the tube is exposed to various stresses and forces in the longitudinal and transverse directions. Mechanically stable tubes withstand these influences, are tear-resistant and are characterised by a corresponding durability. It is useful if the tube is mechanically stable during a process to the extent that it does not have to be replaced several times, but is at least durable for the duration of the process and does not have to be replaced.

The properties of flexibility and mechanical stability are also determined by the choice of material for the tube. The material of the tube is selected, for example, from silicone, polyvinyl chloride (PVC), rubber, in particular natural rubber, synthetic rubber, in particular vulcanised rubber, acrylonitrile butadiene rubber (also known as nitrile butadiene rubber, NBR), acrylonitrile butadiene rubber (NBR) with ethylene propylene diene rubber (EPDM), chlorobutadiene rubber (also known as chloroprene rubber or CR neoprene, Neoprene®), Teflon, polyetheretherketone (PEEK), polyethylene (PE) and/or polyamide.

The shape of the tube is not further limited, any type of tube suitable for the process can be used.

Fluid is to be supplied to the flexible tube. For this purpose, a rigid fluid line is provided above the liquid level of the liquid medium in the reactor, to the end of which the tube is to be attached. The fluid line is either specially set up for this purpose in the reactor or is already present. For example, in a bioreactor for the cultivation of organisms, it is regularly customary to provide fluid supply lines for supplying the organisms with air or oxygen, so that a supply line for foam destruction and/or control is also readily available. For example, the overhead gas flow can also be used for this purpose in a cultivation reactor. In the SUBs, such fluid supply lines are already pre-installed before commissioning.

The term “rigid” for the fluid line shall be understood to mean a fixed, non-movable line. A “rigid fluid line” means that it should be stationary and immovable, while the tube attached to it should be flexible and thus free to move except at the location where the tube is fastened. The material for the fluid line is not particularly limited as long as it is suitable for the supply of compressed fluid. This can be plastic, metal, in particular stainless steel, or glass.

The fluid line is connected to a fluid connection, which is in particular located outside the reactor. The fluid connection is provided for the supply of a compressed fluid which is not particularly restricted, provided it does not adversely affect the foam-generating method in the reactor. The fluid is selected from a gas which is, for example, compressed air, compressed oxygen or a compressed inert gas, such as nitrogen or a noble gas, in particular argon. The fluid may be also selected from a liquid such as water, organic solvent(s), the liquid medium used in the reactor or mixtures thereof. The type of compressed fluid is selected according to the chemical, physical, biological, biochemical, in particular biotechnological or biopharmaceutical method taking place in the reactor. For example, in a biochemical method, in particular a cultivation of cells or microorganisms, it is advantageous if air or oxygen is used as the compressed gas, since the supply of air or oxygen to the cells or microorganisms may be necessary during the cultivation itself.

The hereby defined “compressed fluid” is a fluid of the gas(es) and/or liquid(s) defined above having a pressure that is sufficient to cause the tube to move chaotically under operating conditions, esp. during cultivation of cells. According to one embodiment, the pressure of the fluid in a bioreactor is selected to be at least greater than the pressure in the headspace of the bioreactor. In steel production reactors, for example, the normal operating range for the pressure in the headspace of the reactor may be 0.25±0.1 bar, so that the compressed fluid has a pressure greater than 0.25±0.1 bar. In an alternative embodiment, a reactor, e.g. a bioreactor, is operated at 0 bar; in this case, a minimum pressure of e.g. 0.05 bar can be set for the compressed fluid. A low limit for the pressure for a single-use bioreactor (SUB), which varies significantly from design to design, can be given as an example at about 0.05 bar.

The rigid fluid line is therefore arranged in the liquid-free upper part or head section of the reactor, in particular a chemical reactor, bioreactor including fermenter or another kind of reactor.

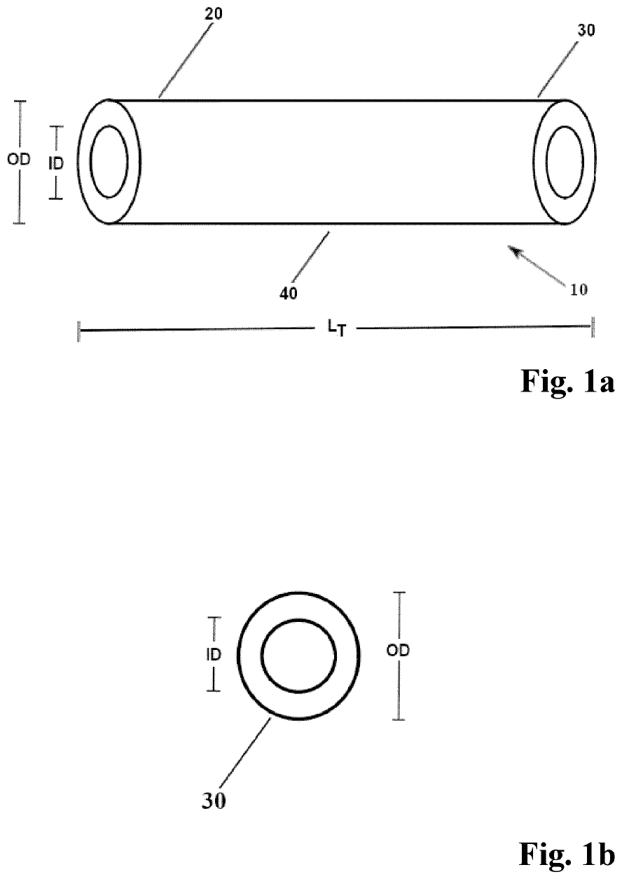

The flexible tube is fastened to the rigid fluid line with one of its two ends and connected to it so that the compressed fluid can be passed through the tube. An exemplary embodiment of a tube is shown in FIGS. 1a and 1b. FIG. 1a shows a tube 10 with a predetermined length L_{T}, an inner diameter ID and an outer diameter OD, as well as a first end 20, a middle section 40 and a second end 30. It is understood that the inner diameter and outer diameter usually remain the same size over the entire length of the tube. FIG. 1b shows a schematic top view of the second end 30 of the tube 10 of FIG. 1a.

The first end of the tube (in FIG. 1a: end 20) is connected to the rigid fluid line and thus attached to it, in particular in a fluid-tight manner, i.e. the flexible tube is fastened to the end of a rigid fluid line. This can be done by any type of attachment for a flexible tube to a line. For example, the tube can simply be pushed onto the fluid line and, if the tube and fluid line have a suitable diameter, it already seals the line in a fluid-tight manner and is fastened there in a suitable manner. However, an additional fastening means can also be used for this purpose, such as a hose clamp, or adhesive bonding or the like.

It has been found advantageous if the attachment point of the first end of the tube to the fluid line is arranged on the central axis through the reactor in the liquid-free upper part of the reactor. In this way, the fluid line and thus also the tube attached to it are centrally located above the liquid level of the liquid medium to be filled in or already present and can thus easily reach the entire foam layer formed on the liquid medium in order to destroy or control it. Alternatively, the fluid line and thus also the tube can be mounted offset from the centre in a distance from the central axis or in or on a side wall of the reactor.

The second end of the tube (in FIG. 1a: end 30) is not fixed but is free to move. When no fluid is flowing through the tube, i.e. before the tube is put into operation, when it is in the rest position, it hangs loosely down from the attachment point on the fluid line into the reactor. If the attachment point of the fluid line and tube is located on the central axis of the reactor, the tube will therefore hang down through the reactor on the central axis.

As an alternative, the length L_{T} of the tube is chosen so that the second end of the tube comes into contact with the liquid level of the liquid medium. The expression “the second end of the tube comes into contact with the liquid level of the liquid medium” means that one end of the tube contacts the liquid surface before (out of function) and at least temporarily during the process of foam reduction and/or control and, in particular, may also be immersed in the liquid medium. This immersion in the liquid medium may be, for example, to a small extent. According to some embodiments, a minimal immersion may be advantageous. In this context, it must be taken into account that the filling level of the reactor with liquid medium will generally not be constant, the filling level may vary by a few percent during the reaction taking place in the reactor, for example. For example, there may be a maximum fill level that varies by about ±10%. This may result from reactants, liquid medium or the like being added and liquid ingredients which may be removed. In cultivation methods, for example, nutrients may be added continuously and waste medium may be removed. The exact immersion depth of the tube in the liquid medium cannot therefore be stated without further ado and depends on each individual case. However, this is not important, provided that the process for destroying foam and/or controlling the foam height H_{F} in a reactor is not impaired by this. Furthermore, if necessary, contamination can generally be excluded in the case of direct contact with the liquid medium by sterilizing the tube before use. This avoids that the process of foam destruction and control can be adversely affected.

According to another alternative the length L_{T} of the tube may be selected so that the second end of the tube can come into contact with foam formed on the liquid medium, but cannot come into contact with the liquid medium in the reactor. In this case the tube therefore ends above the liquid level of the liquid medium present in the reactor, with the second end not touching the liquid level of the liquid medium. This can avoid disturbance of the liquid phase in the reactor and possible shear forces. Selecting the tube length in such a way that the tube can come into contact with the foam but not with the liquid medium therefore has the advantage that only the foam is destroyed by the uncontrolled movements of the tube, but the liquid medium is not affected by this. This variant can be advantageous for particularly sensitive reaction methods or products.

The first end of the tube is therefore the only attachment point for the tube. In case the second end does not come into contact with the liquid medium, the tube is completely outside the liquid medium over its entire length. The distance between the second end of the tube and the liquid level or the maximum height of the liquid in the reactor before the tube is put into operation therefore corresponds to the maximum foam height H_{F(max)}, which is allowed in the reactor. The maximum foam height H_{F(max)} represents the distance between the liquid level and the highest point of the foam formed in the reactor. The foam height H_{F} is the height of the foam measured at a certain point in time during a foam-generating method. The maximum foam height H_{F(max)} is the maximum level of the foam allowed when the process or device according to the invention is used. If the liquid level in the reactor is not constant, a maximum foam height H_{F} can possibly only be estimated.

As soon as the method taking place in the reactor forms foam, active foam destruction and/or control, i.e. the device or process of the invention, is started. This is done by adjusting the fluid flow through the tube so that the second end of the tube performs a chaotic movement pattern in the reactor and by contact of the tube with the foam and by the compressed fluid exiting the tube, the foam formed is destroyed and/or the foam height H_{F} of the foam formed is controlled.

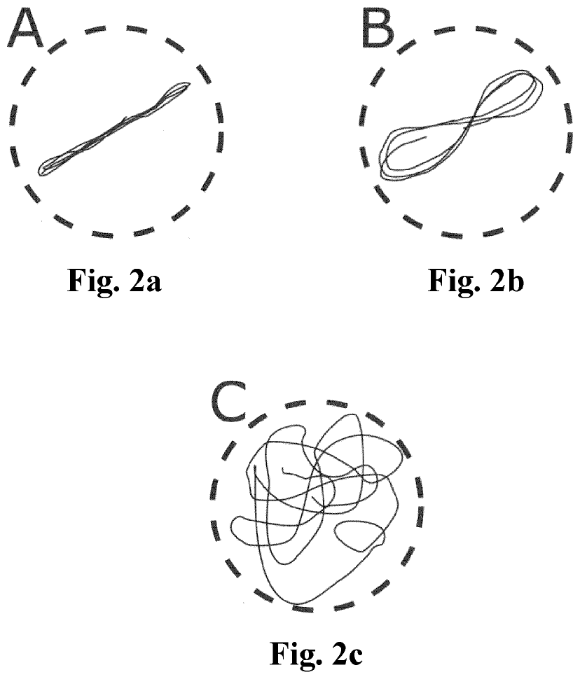

The fluid flow in the flexible tube sets it into chaotic motion. The chaotic movement is most easily illustrated by a graphical representation. Basically, three movement patterns can be distinguished for the tube movement. The three movement patterns are exemplarily and schematically shown in FIGS. 2a, 2b and 2c in a top view of the second moving free end of a tube by a fluid flow.

It is shown an example of a linear movement in FIG. 2a, an example of a periodic or periodically similar movement in FIG. 2b and an example of a chaotic movement in FIG. 2c. A chaotic movement pattern, as shown in FIG. 2c, is therefore non-linear and not periodic, but unpredictable. The chaotic movement itself is irreversible, irregular and statistically random. The chaotic movement is a stochastic movement, whereby the random principle is applied. For effective foam destruction or control, chaotic motion as shown in FIG. 2c is required. This phenomenon of the chaotic movement of a tube is familiar to anyone who has ever had an opened garden hose lying on the lawn and then turned on the water, whereby at high water pressure the tube begins to dance and one can only get hold of it with difficulty.

Therefore, as soon as the method in the reactor starts to form foam, the fluid connection is opened and the pressurised fluid is fed through the tube. The fluid velocity is adjusted so that the second free end of the flexible tube performs a chaotic pattern of movement in the reactor due to the fluid flow. The skilled person can achieve this in a simple way by increasing the fluid flow through the tube.

If the length of the tube is selected so that the second end does not contact the liquid medium then the selected length L_{T} of the tube determines or adjusts the maximum foam height H_{F(max)} that can occur during the foam-generating method. The set tube length therefore depends on a number of parameters, in particular the dimensions of the reactor and the filling level in the reactor, taking into account a varying filling level if necessary, and the maximum foam height H_{F(max)} to be set in the reactor that is acceptable for the method. Since a method takes place in the reactor while medium can be added and/or removed, continuously, for example in a batch process, the expected final volume should be taken into account. The length L_{T} of the tube can therefore be suitably selected by the skilled person in each individual case.

The adjusted tube length therefore depends on a number of parameters, in particular the dimensions of the reactor and the filling level in the reactor, taking into account a varying filling level if necessary, and the maximum foam height H_{F(max)} to be set in the reactor which is acceptable for the process.

The destruction or control of the foam is therefore carried out by means of a targeted pressurised fluid blast or exit pulse of the compressed fluid at the end of the tube, which on the one hand causes the tube movement and on the other hand the foam destruction. On the one hand, therefore, the foam is irradiated with pressurised fluid by means of a flexible and, in particular, mechanically stable tube that moves chaotically and, on the other hand, the tube movement leads to the destruction of the foam where it comes into contact with the tube.

The flexible tube is set in motion by the flow of the fluid. As it is a chaotic movement, the tube moves over the entire foam surface. The uncontrolled moving tube comes into contact with the formed foam and destroys it, depending on how long the tube was chosen. The chaotic movements of the tube and the fluid escaping under pressure therefore destroy the foam on contact and thereby control the foam height accordingly. A propagation of foam with an increase in the foam layer formed or even an overfoaming of the reactor is thus prevented.

With regard to an economical mode of operation, it is expedient if the amount of fluid required for the operation of the tube is kept as small as possible. This can be achieved, for example, by selecting suitable parameters and materials for the tube. The dimensions of the tube are generally determined by 3 parameters: inner diameter, outer diameter, length. The wall thickness is already determined by the inner diameter and outer diameter, whereby the following applies: (outer diameter-inner diameter): 2=wall thickness.

The length of the tube, the thickness of the tube wall and the inner and outer diameters of the tube can be varied. The fluid volume flow is then adjusted so that the tube can move freely in a chaotic movement pattern due to the momentum force of the fluid volume flow. To achieve a low fluid flow rate, the selected tube length can be chosen depending on the dimensions of the reactor and the height of the liquid level, as well as the tube wall thickness and the tube diameter.

The movement of the tube is also influenced by the material chosen for the tube, in particular the elasticity of the tube, and the weight of the tube. The material chosen therefore determines the flexibility of the tube, whereby the more flexible the tube, the less fluid is needed to set the tube in chaotic motion.

Two parameters may be relevant for the flexibility of the tube: the wall thickness and the inner diameter of the tube. As a rule of thumb, one can state that the stiffer the material of the tube, the smaller the wall thickness is selected to achieve the chaotic movement pattern. The inner diameter of the tube and the fluid flow rate determine the fluid exit velocity and thus the momentum that moves the tube. Depending on wall thickness, the smaller the inner diameter of the tube, the lower the fluid flow rate can be set to move the tube chaotically.

The compressed fluid burst or exit momentum of the compressed fluid at the end of the tube, which leads to the chaotic movement of the tube, is thus determined by the fluid exit velocity, for which the fluid pressure and the internal diameter of the tube are relevant. “Fluid exit velocity” here means the fluid flow velocity at the exit end from the tube (in FIG. 1 the second end 30). The fluid exit velocity can be calculated from the fluid pressure and the internal diameter of the tube, so that the person skilled in the art is able to set a suitable fluid exit velocity on the basis of a few tests.

The fluid flow rate, e.g. the gas flow rate, is used in the present disclosure as synonymous with the gas volume flow, e.g. the gas volume flow, which is understood to mean the volume of fluid, for example gas, per unit time through a specified cross-section. For example, the gas flow rate is given in [m³ s⁻¹]. The gas flow rate is related to the average gas flow velocity over the cross-sectional area, so that it is possible to draw reciprocal conclusions from one to the other. In addition, the pressure, gas flow rate and gas flow velocity depend on each other due to the generally known ideal gas law. Furthermore, the superficial gas velocity describes

$${w}_{G 0} = \frac{q}{A}$$

the nominal velocity at which the gas phase would move in an empty reactor. The superficial gas velocity is an important parameter for the fluid mechanical description of multiphase systems (gas/liquid). Assuming that the fluid in form of gas entering the reactor is homogeneously distributed over the cross-sectional area of the reactor and that all the fluid is converted into foam, the superficial fluid velocity corresponds to the foam formation rate. These basic physical laws are known to the skilled person in the prior art.

The weight of the tube has already been mentioned as another parameter for the tube. The weight of the tube is expressed in particular in the rigidity (bending stiffness) of the tube. If the rigidity is too high, it may be necessary to pass more fluid through the tube. However, this is usually not desirable. To reduce the fluid flow rate, the weight of the tube can then be reduced, for example. This can be done by selecting the material for the tube or varying the parameters for the tube, for example the choice of wall thickness or tube length. The weight G can also be readily calculated from the dimensions of a tube by determining the volume V of the tube as a hollow cylinder by

$$V = π \times {L}_{T} \times {( {d}_{2} - {d}_{1} )}^{2}$$

and from this then the weight G of the tube is calculated with:

$$G = V \times ρ$$

wherein
V volume of the tube [m³]
L_{T} length of the tube in [m].
d₂ outer diameter (OD) in [m]
d₁ inner diameter (ID) in [m]
TT circle number or Archimedes' constant (pi)
G weight of the tube [kg]
ρ (roh) Density of the tube material in [kg/m³].

Example parameters for the tube can be selected from the following ranges without limiting them to this:
Outer diameter from 1.0 mm to 15.0 mm,
Inner diameter from 0.5 mm to 5.0 mm and
Wall thickness from 0.25 mm to 7.25 mm.

It is not practical to give any combinations of possible parameters for the tube, as these depend on numerous variables and parameters, such as, among others, the local conditions (e.g. space available, location and type of fluid connection and the like), the parameters and geometry for the reactor, the parameters of the foam-generating method to be controlled, the size of the method approach and the like. However, the indicated value ranges can be used for orientation. In particular, commercially available tubes can be used, so that the commercially available tubes with the respective available dimensions can be used without further ado.

It may be useful for the tube to have a minimum length so that the second end of the tube can move freely. The minimum length can be about 100 mm, depending on the material of the tube. Shorter tube lengths—depending on the material selected—are also possible, but may then require very high fluid flow rates, which are disadvantageous and therefore undesirable. A maximum length of the tube cannot be given, as this depends on the individual case.

According to one embodiment, it is possible to use not only one tube in the reactor, but several tubes at the same time, which can be distributed over the liquid-free upper part of the reactor. In particular, the multiple tubes are provided in such a way that they do not interfere with each other while performing their respective chaotic motion. For example, it would be conceivable to provide several tubes on fluid lines in the head portion or on the side walls of a reactor, with the same or different length and/or the same or different diameters, each fulfilling its function without interfering with each other.

The number and position of the tubes depends on the reactor geometry. Thus, according to the invention, a wide variety of embodiments is possible, wherein the one or more tubes are installed in the head portion of the reactor, but not in the side walls, or the one or more tubes are installed in or near the side walls, wherein a stirrer which is present can be driven from above or from below.

In a configuration with a stirrer driven from below, a single tube installed, for example, centrally in the reactor, e.g. with the attachment point lying on the central axis of the reactor, may be advantageous. According to an embodiment, the attachment point of the tube may be present in each case at a distance from the side wall of the reactor that has the same size everywhere.

According to another embodiment a single tube may be installed offset from the centre or installed in or near a sidewall of the reactor wherein a stirrer is, for example, driven from below.

According to another embodiment in which a stirrer is driven from below at least two tubes may be installed in the reactor, particularly installed in the head portion, but not the side walls, of the reactor. In this case, it may be advantageous if the at least two tubes are spaced apart from each other in the head portion and the lengths L_{T} of the at least two tubes are selected in such a manner that the tubes do not contact each other during their chaotic movement and do not contact the side walls.

According to another embodiment in which a stirrer is driven from below at least two tubes may be installed in the reactor in or near the side walls of the reactor. In this case, it may be advantageous if the at least two tubes are in particular installed opposite one another in the reactor whereby the lengths L_{T} of the at least two tubes are selected in such a manner that the tubes do not contact each other during their chaotic movement

In the case of a top-driven stirrer, it would be convenient if, for example, at least two individual tubes are provided at a suitable distance from each other at the same or different height in the reactor. For example, 2 tubes could be provided at the same height in the reactor, with the two attachment points of the two tubes to the two rigid fluid lines being provided at a distance from each other equal to or greater than the sum of the length of both tubes. This may be convenient so that the two tubes do not interfere with each other during their chaotic movement. In particular, the distance could correspond to the diameter of the reactor, i.e. the two tubes could be fixed opposite each other in the side wall at the same height. For example, the at least two tubes are installed in the side walls opposite one another in the reactor whereby the lengths L_{T} of the at least two tubes are selected in such a manner that the tubes do not contact each other during their chaotic movement and do not contact the stirrer rod of the stirrer. This would allow both tubes, which are set into chaotic motion, to destroy or control the foam layer that forms.

According to a further embodiment in case of a top-driven stirrer at least 2 tubes, particularly at least 3 tubes or at least 4 tubes may be installed in the head portion but not the side walls of the reactor. In this case, it may be advantageous if the tubes are spaced apart from each other in the head portion so that the tubes do not interfere with each other in their chaotic movement, do not interfere with the stirrer rod and do not contact the side walls during their chaotic movement. This is achieved, for example, by spacing the tubes apart from each other and/or selecting the tube length L_{T} of each tube in such a way that a tube cannot come into contact with the stirring rod coming from above and cannot come into contact with another tube and the side walls during the chaotic movement.

The expression “the tube is installed in the reactor” or equivalent terms means that the flexible tube is attached to the end of a rigid fluid line extending into the reactor. The expression “a tube is installed in the head portion but not in the side walls of the reactor” is to be understood in the sense that a tube is attached to the end of a rigid fluid line provided in the top part of the reactor, in particular in the cover of the reactor, and extends from the cover into the reactor but not from the sidewalls. The expression “a tube is installed in or near the side walls of the reactor” is to be understood in the sense that a tube is attached to the end of a rigid fluid line which is provided in the side walls of the reactor and extends from the side walls into the reactor.

If more than 2 tubes are to be used, it is advantageous if they are distributed in the liquid-free part of the reactor in such a way that they do not touch each other during the execution of the chaotic movement pattern and thus cannot become entangled.

It goes without saying that a rigid fluid line is provided for each tube in the reactor.

For example, to select the tube length, tube diameter, tube wall thickness, tube material, and fluid flow rate for a particular reactor and foaming process, the tube parameters may first be determined and then the fluid flow rate gradually increased until the tube moves in a chaotic pattern. Optimisation of the individual parameters, and in particular a reduction in the fluid flow rate, can then be achieved by varying the parameters of the tube in accordance with the explanations in the present disclosure.

As a rule of thumb, the gas flow rate for a tube performing a chaotic movement can be in the range of 0.00001 to 0.001 m³/s or 0.00003 to 0.001 m³/s or 0.00005 to 0.001 m³/s or 0.00008 to 0.001 m³/s. However, other gas flow rates are also possible and, as already explained, depend in particular on the internal tube diameter and the tube length.

It was also found in experiments that the gas flow rate does not represent a continuously increasing or decreasing magnitude depending on the tube length. For this purpose, the minimum necessary gas flow rate was determined experimentally for different tube diameters and tube lengths and the gas flow rate was measured that is at least necessary for the tube to move chaotically. Although the tube already starts moving at relatively low gas flow rates, it does not always move chaotically, but can also initially move only linearly or periodically. In order to achieve a chaotic movement, the gas flow rate is then simply increased in this case.

In the experiments, it was found that a small diameter of the tube requires only a low minimum gas flow rate Vₘ for a chaotic movement of the tube to be achieved, almost regardless of the selected length of the tube. For larger diameters of the tube, a significantly higher minimum gas flow rate Vm is measured for the respective lengths of the tube, which is required for a chaotic movement of the tube to exist. Therefore, the smaller the inner diameter of the tube, the lower the gas flow rate or gas volume flow is required to move the tube chaotically. The minimum gas flow rate required to create chaotic movement of the tube therefore increases as the diameter of the tube increases. The inner diameter of the tube and the gas flow rate therefore determine the gas exit velocity and thus the momentum that moves the tube, as explained earlier. The findings are analogously applicable to liquids.

If, for a given length of tube, there is no chaotic movement at the set fluid flow rate in a reactor, one can therefore, for example, either reduce the inner diameter of the tube or increase the fluid flow rate until there is chaotic movement of the tube. The latter variant is less favourable for economic reasons.

Furthermore, it was found that the minimum gas flow rate required to perform a chaotic movement of the tube first decreases and then increases with increasing tube length (see example 1 and the associated FIG. 4 for more details). This is attributed to the fact that for short or shorter tube lengths the inertia forces dominate and for long or longer tube lengths the weight forces dominate. The inertia forces represent the persistence of the tube and in principle also describe its rigidity.

Experiments were therefore carried out to better verify the influence of inertial forces and weight forces on the different tube lengths and diameters and also to be able to quantify them. For this purpose, the force ratio (quotient) of momentum force F_{I} to weight force F_{G} was examined for different tube lengths and diameters.

The following formula (1) applies to the momentum force F_{I}:

$${F}_{I} = ρ * {u}^{2} * A , \qquad ( 1 )$$

wherein
F_{I} momentum force in [N]
ρ (rho) density of the tube material in [kg m⁻³]
u gas exit velocity in [m s⁻¹]
A cross section of the tube in [m²]

The following formula (2) applies to the weight force:

$${F}_{G} = ρ * π / 4 * ( {d}_{2}^{2} - {d}_{1}^{2} ) * g \qquad ( 2 )$$

wherein
F_{G} weight force in [N]
ρ (rho) density of the tube material in [kg m⁻³]
π circle number or Archimedes' constant (pi): 3.14159265 . . .
d₂ outer diameter (OD) of the tube in [m]
d₁ inner diameter (ID) of the tube in [m]
g acceleration due to gravity in [m s⁻²].

The force ratio F_{I}/Fe is a dimensionless value.

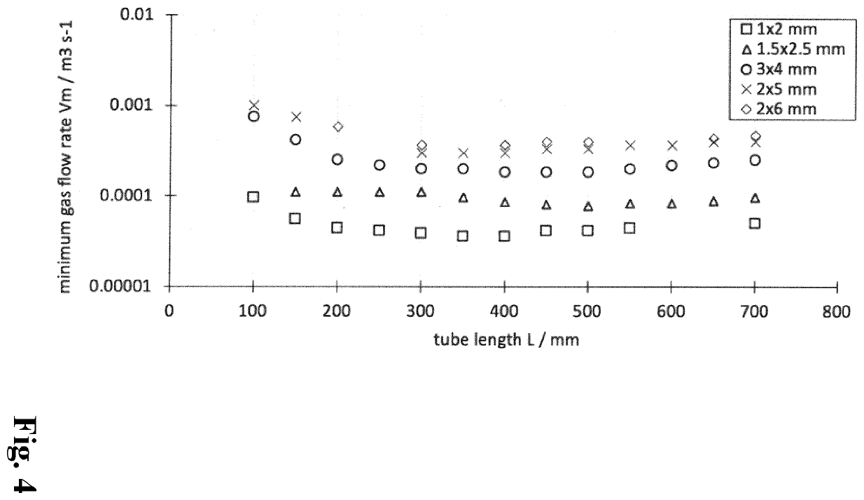

The experiments and their results are described and illustrated in detail in example 2 and the associated FIG. 5. It was found that significantly greater momentum forces are required for short tube lengths, while an almost constant ratio F_{I}/F_{G} of approx. 0.13 is achieved with a longer tube length. From this it can be concluded that particularly good foam destruction is achieved when:

$${F}_{I} / {F}_{G} < 10$$

$$or$$

$${F}_{I} / {F}_{G} < 5 or {F}_{I} / {F}_{G} < 4 or {F}_{I} / {F}_{G} < 3 or {F}_{I} / {F}_{G} < 2 ,$$

especially if applies:

$${F}_{I} / {F}_{G} < 1 .$$

According to one embodiment, in particular, a force ratio F_{I}/F_{G}<1 was found to be advantageous. At a ratio of <1, the weight force outweighs the momentum force so that more “force” can be used to break the foam. This is unexpected for the skilled person, as one would assume that the momentum force should always be greater than the weight force in order to obtain a chaotic movement pattern of a tube. However, it is the other way round, which is completely surprising.

With the experiments it was also found that the better the mobility of the tube, the better the foam destruction and/or control. In other words, the better the flexibility of the tube, the more effectively the tube works. For example, selecting a more flexible and more lightweight material for the tube will thus result in the active foam destruction and/or control working better, and then, in particular a lower minimum fluid flow rate can also be set in order to maintain the chaotic movement of the tube.

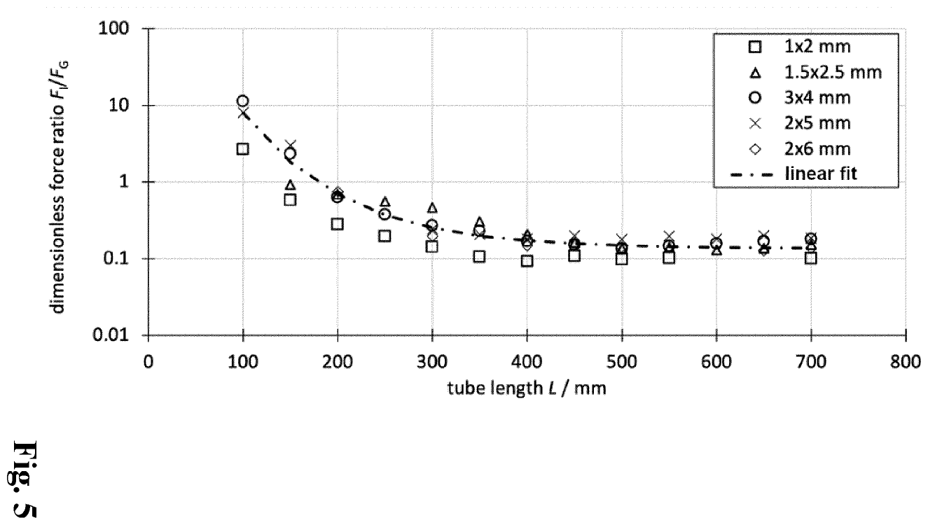

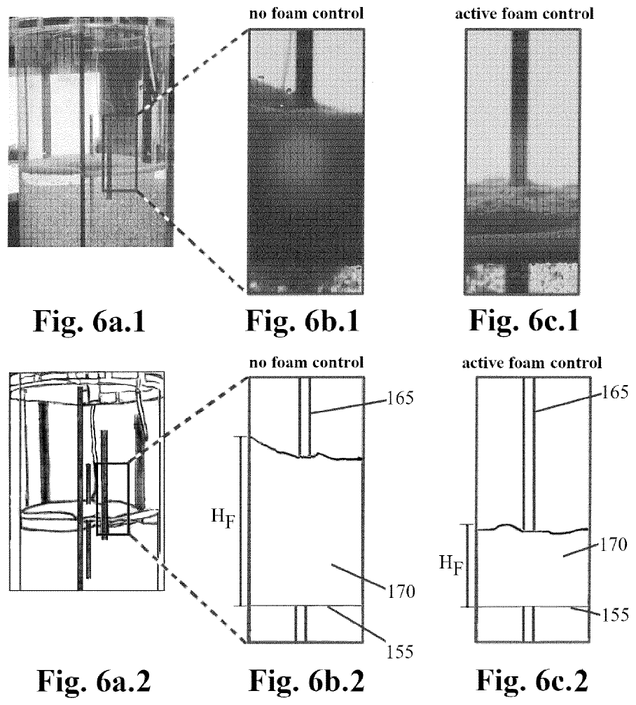

Furthermore, experiments were carried out to investigate the effect of the device or the process of the invention on foam formation. Details are explained in the examples 3 to 6 and the associated FIGS. 6a.1 to 9. It was found that if there is no active foam control (without device or process according to the invention), the foam very quickly takes up a large part of the reactor content and very soon fills it completely. In contrast, if active foam control is carried out using the device or process of the invention, then a significantly reduced foam height H_{F} can be obtained and maintained throughout the process. Destruction of the excessively formed foam and control to a foam height H_{F} that does not impair the foam-generating method in the reactor takes place.

A comparison of the foam growth in a reactor over the course of a foam-generating method shows, for example, a clearly increasing foam quantity over the course of the method when no foam control takes place and a controlled foam height when active foam control (with device or process according to the invention) takes place, whereby the foam height can be kept almost constant at a very low level.

The invention is also directed to a method (a foam-generating method) for culturing prokaryotic or eukaryotic cells in liquid cell culture in a bioreactor, wherein foam is generated during the method, wherein a foam destroying device according to the present invention is used in the bioreactor performing a process for destroying foam and/or controlling the foam height H_{F} in the reactor according to the present invention.

According to an embodiment, the foam-generating method is an in vitro method for the cultivation of cells and particularly involves the use of eukaryotic, preferably mammalian cell lines used for high expression of a product, such as a heterologous protein or a recombinant virus, expressed e.g. from non-integrating or integrating vectors comprising respective coding sequences and optionally further genetic elements e.g. for selection and/or regulation. With respect to background information on the cultivation of cells, detailed information on mammalian cells, heterologous proteins or recombinant viruses, reference is made to WO 2021/165302 A1, the contents of which are incorporated by reference in their entirety into the present disclosure. In particular, reference is made to paragraphs [0081] to [0088].

Example prokaryotic cells are bacteria like e.g. Bacillus subtilis and Escherichia coli. Example eukaryotic cells are yeast cells, preferably Picha or Saccharomyces, or insect cells. Example mammalian cells are human and rodent cells, preferably from rat, mouse and chinese hamster ovary (CHO). Heterologous proteins are for example pharmaceutically useful proteins like growth factors, hormones or monoclonal antibodies or antibody-derived formats like IgG, nanobodies, diabodies, zweimabs etc., in principle known to the person skilled in the art.

It is also disclosed a method (a further foam-generating method) for producing a recombinant protein, the method comprising the steps of:
step a) culturing prokaryotic or eukaryotic cells expressing a recombinant protein in cell culture in a bioreactor, wherein foam is generated during culturing of the cells;
step b) harvesting the recombinant protein;
step c) purifying the recombinant protein;
wherein in step (a) a foam destroying device according to the present invention is used in the bioreactor performing a process for destroying foam and/or controlling foam height H_{F} in the reactor according to the present invention.

The recombinant protein of the above method of the invention is produced in eukaryotic cells, following expression, the recombinant protein is harvested and further purified. The recombinant protein may be recovered from the culture medium as a secreted protein in the harvested cell culture fluid (HCCF) or from a cell lysate (i.e., the fluid containing the content of a cell lysed by any means, including without being limited thereto enzymatic, chemical, osmotic, mechanical and/or physical disruption of the cell membrane and optionally cell wall) and purified using techniques well known in the art, esp. affinity chromatography, preferably via protein A, anion- and/or cation exchange chromatography and further optional steps like e.g. virus inactivation and one or more filtration and concentration steps like diafiltration and ultrafiltration. The recombinant protein may then be formulated into a pharmaceutically acceptable formulation suitable for administration, for example in a dry (powder) formulation, liquid buffered formulation for oral, inhalatory, subcutaneous, intravenous or other administration route to a human or animal patient.

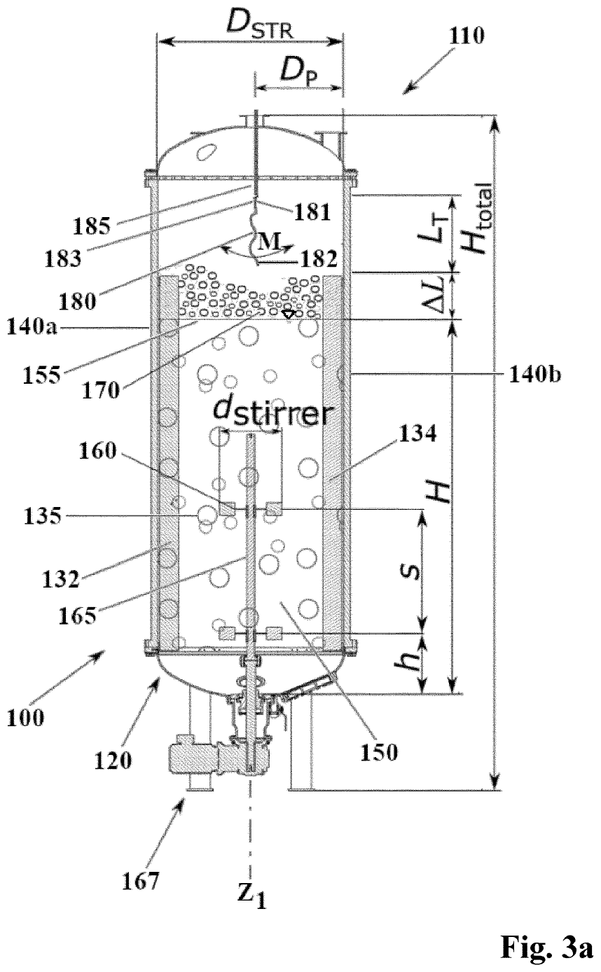

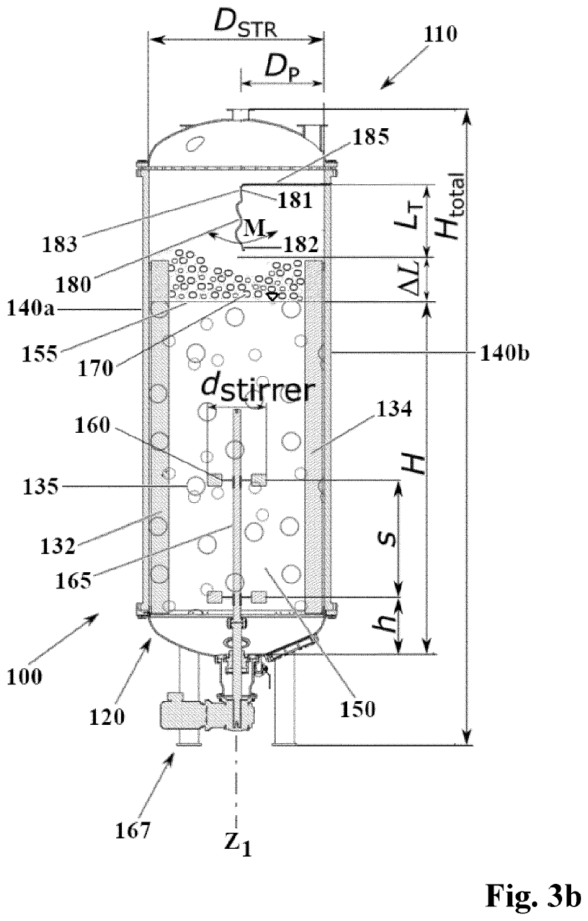

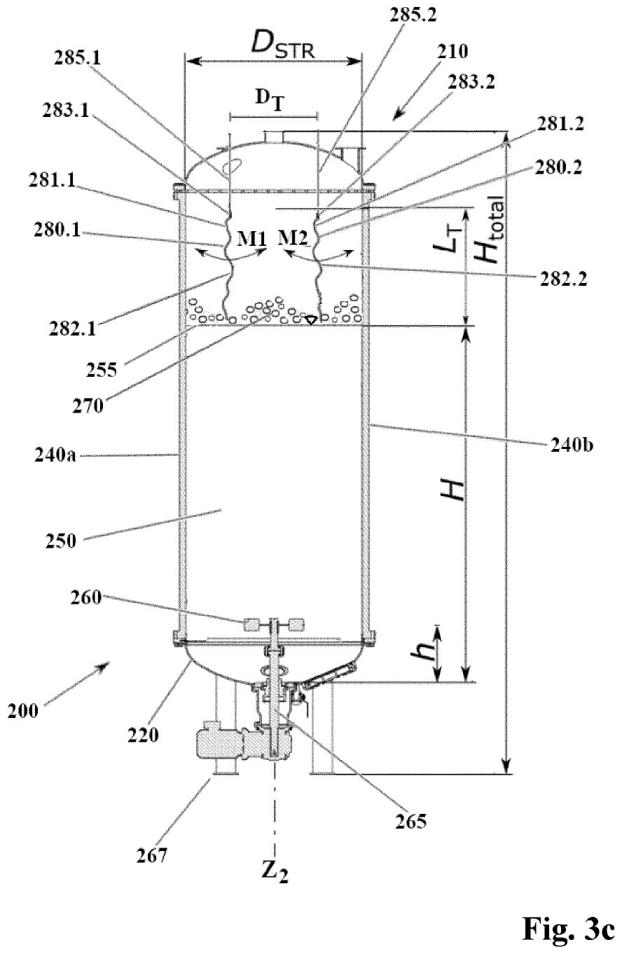

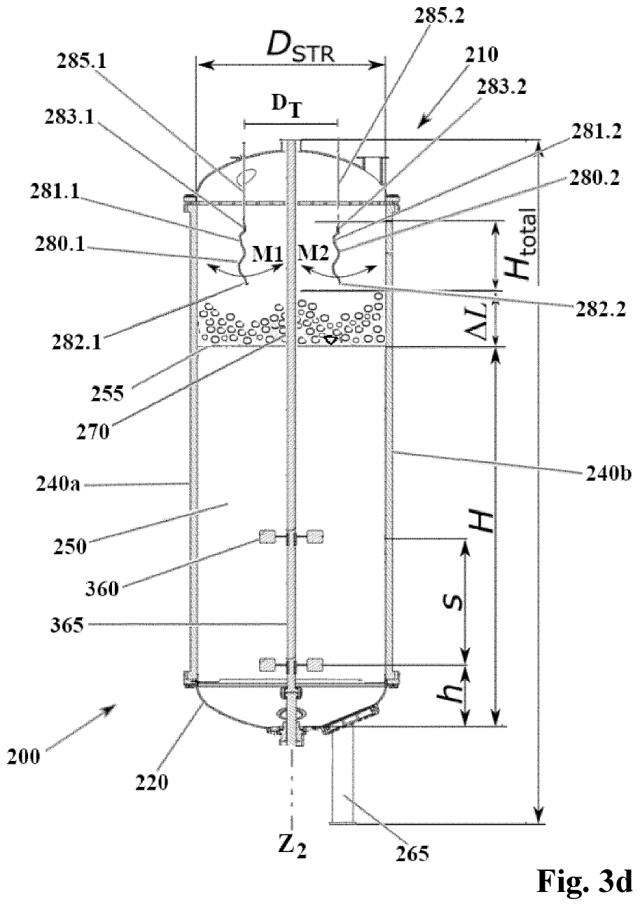

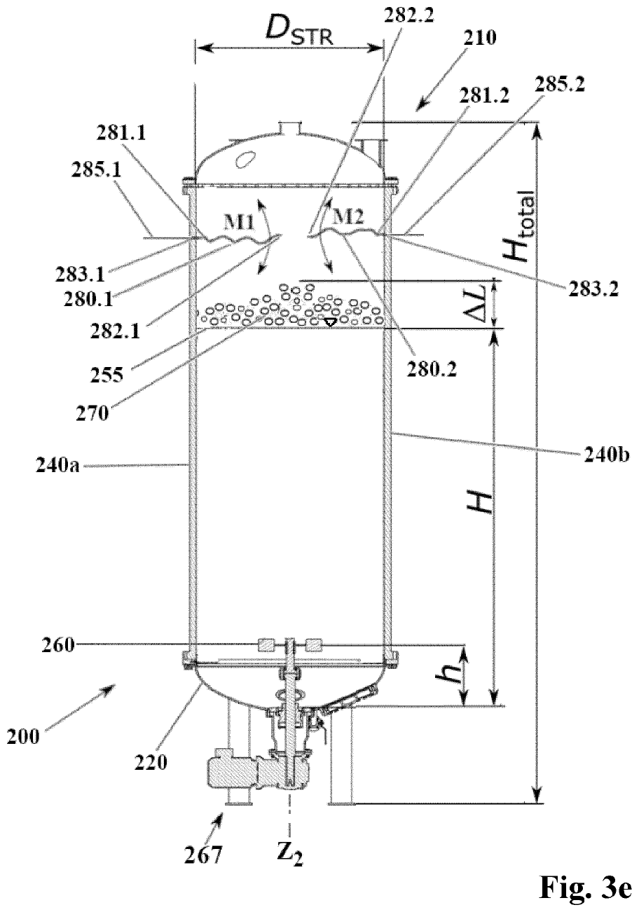

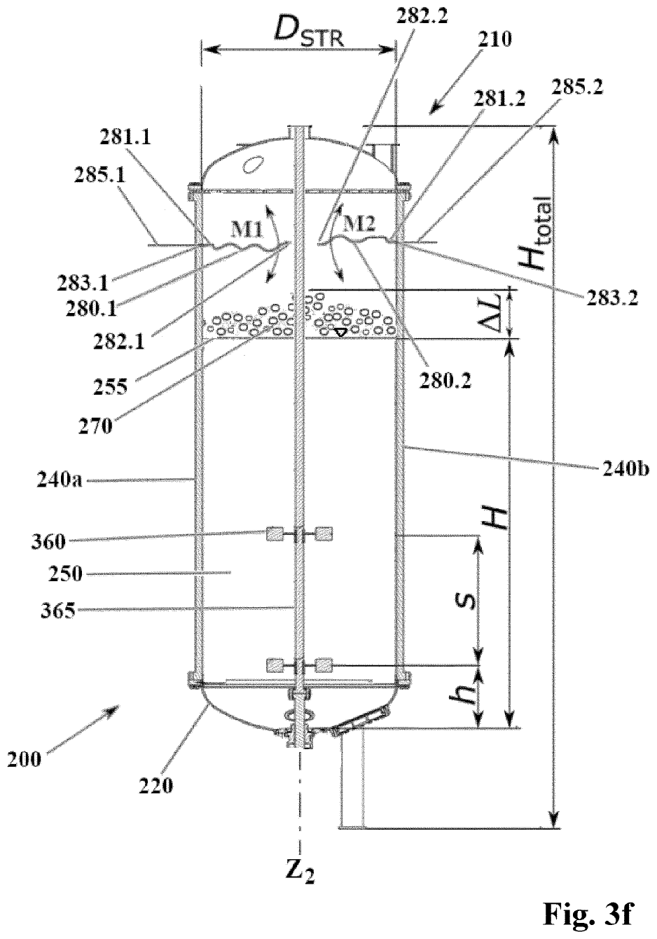

In the following, embodiments of the present invention will be described in detail with reference to FIGS. 3a to 3f, without limiting the invention thereto.

FIG. 3a shows a sectional view through a reactor in the form of a bioreactor 100 according to an embodiment of the invention, which is shown in schematic simplified representation. The bioreactor 100 shown is used for the cultivation of cells. Nutrients are administered to the culture for cultivation (not shown) and waste liquids is removed (not illustrated). In the example case shown, where aerobic cells are cultivated, oxygen is provided by technical means like spargers (not shown) illustrated by the gas bubbles 135. Two baffles 132, 134 are respectively arranged on the side wall 140a, 140b of the bioreactor 100 opposite each other. The bioreactor 100 is in the form of a cylindrical tube with a reactor diameter D_{STR} and a total height Hₜₒₜₐₗ having a top part 110 and a bottom part 120. The bioreactor 100 may be made of stainless steel and can be reused several times for cultivation. The bioreactor 100 could also be a single-use bioreactor (SUB). The bioreactor 100 shown may be used for large-scale production.

The cells cultivated in the bioreactor 100 are, for example, eukaryotic cells such as Chinese hamster ovary (CHO) cells or yeast cells, and can be used, for example, to produce antibodies for therapeutic use or produce other useful substances.

The bioreactor 100 already comprises a liquid medium 150 in which the cells (not shown) are suspended. A stirrer 160 is provided, in this case a Rushton turbine with a diameter dₛₜᵢᵣᵣₑᵣ with a dual impeller system, the lower impeller being mounted at a distance h (stirrer bottom clearance) from the bottom 120 of the bioreactor 100 and the two impellers being mounted on a stirrer rod 165 and spaced at a distance s from each other. The stirrer 160 is driven by a stirrer motor 167 mounted from below and located outside the reactor 100. The liquid medium 150 is present in the bioreactor 100 up to a height H with a liquid level 155. The liquid medium 150 is not further limited, but can be any suitable liquid medium that can be used for the cultivation of cells.

In the bioreactor 100 shown, a cultivation process is already taking place that generates foam 170 that accumulates on the liquid medium 150. Foam destruction and/or control is already taking place in the bioreactor 100 shown. For this purpose, a flexible tube 180 is in chaotic motion, symbolised by the curved double arrow M. One end, here: the first end 181, of the tube 180 is fastened to a rigid fluid line 185 provided in the upper part or head portion 110 of the bioreactor 100, but not in the side walls 140a or 140b which leads to a fluid connection (not shown). The other end 182 of the tube 180 is not fastened and is free to move. When at rest, i.e. when no compressed fluid is flowing through the tube 180, the tube 180 hangs loosely downwards and has the total length L_{T}. In FIG. 3a, please note that the tube 180 is already in motion (symbolized by the serpentine lines of the tube 180), so the tube 180 shown does not reach the total length L_{T} due to the movements performed (shortening of the tube due to the curvature of the tube during the movement).

The fluid flowing through the tube 180 is compressed air in the example shown, as air is supplied to the culture anyway. However, another gas, such as compressed oxygen or an inert gas or another fluid such as water or liquid medium, could also be used.

The tube 180 is arranged in the head portion 110 at a distance D_{P} from the side wall 140b of the bioreactor 100, which may be half the diameter D_{STR} as shown here. The tube 180 is therefore located in the rest position approximately centrally in the reactor 100 and arranged on the central axis Z₁ of the bioreactor 100. In particular, in this example case, also the rigid fluid line 185 shown, which extends through the reactor lid, the attachment point 183 of the tube 180 to the fluid line 185, and the tube 180 at rest are located on the central axis Z₁ of the reactor 100. Alternatively, the tube 180 could be offset from the central axis Z₁ of the bioreactor 100, or it could be attached in or nearby a side wall 140a or 140b. There could also be multiple tubes at the same time to destroy and/or suitably control the foam 170. The number and position of the tubes will depend, for example, on the reactor geometry. However, in the configuration shown with a stirrer 160 driven from below, a single tube 180 installed approximately in the centre of the reactor is advantageous, with a particularly equal distance from the side walls 140a and 140b, respectively.

The tube 180 is constructed of flexible material, such as silicone, polyvinyl chloride (PVC), rubber, in particular natural rubber, synthetic rubber, in particular vulcanised rubber (rubber), acrylonitrile butadiene rubber (NBR), acrylonitrile butadiene rubber (NBR) with ethylene propylene diene rubber (EPDM), chlorobutadiene rubber (CR neoprene), Teflon, polyether ether ketone (PEEK), polyethylene (PE) and/or polyamide or also another material. It is useful if the material is a mechanically stable material so that the tube 180 does not break down during foam destruction and/or control. The materials specified above usually provide sufficient mechanical stability.

The length L_{T} of the tube 180 is selected in this embodiment in such a way that the tube 180 comes into contact with the foam 170, but not with the liquid medium 150, in particular also the liquid level 155, so as not to impair the cultivation method taking place in the bioreactor 100. The distance ΔL drawn in FIG. 3a indicates the distance of the tube 180 from the maximum height of the liquid or the liquid level 155 in the bioreactor 100. ΔL represents the minimum distance between the second end 182 of the tube 180 and the liquid level 155 and therefore represents and/or adjusts the maximum height H_{F(max)} of the foam. It should be taken into account that the liquid level 155 in the reactor 100 may vary during the process.

According to an embodiment, in particular with regard to an economical mode of operation, the inner diameter of the tube 180 is selected in such a way that the fluid flow rate through the fluid line 185 is as low as possible, but the tube 180 nevertheless performs a chaotic movement pattern. The outer diameter may also be selected in an appropriate manner and has an effect on the wall thickness of the tube 180 and thus, among other things, the flexibility and weight of the tube 180. The material of the tube also plays a role in, among other things, the weight and flexibility of the tube 180. If the material of the tube 180 is selected stiffer, it is expedient to select the wall thickness smaller to achieve the chaotic movement pattern. As a rule of thumb, it can be stated that the more flexible and the lighter the material of the tube 180 and the smaller the inner diameter of the tube 180, the less fluid flow rate is required to set a chaotic movement pattern of the tube 180. In simplified terms, it can be stated that the inner diameter of the tube 180 and the fluid flow rate determine the fluid exit velocity and thus the momentum that moves the tube 180. Thus, the smaller the inner diameter of the tube 180 is selected, the lower the fluid flow rate can be set to move the tube 180 chaotically.

In FIG. 3a, the tube 180 has already started foam destruction and/or control. The destruction or control of the foam 170 takes place by means of a targeted exit pulse of the compressed fluid at the second end 182 of the tube 180, which causes the tube to move and the foam destruction and/or control. On the one hand, therefore, the foam 170 is irradiated with pressurised fluid by means of a flexible and, in particular, mechanically stable tube 180 which moves chaotically and, on the other hand, the tube movement leads to the destruction of the foam 170 where it comes into contact with the tube 180. The uncontrollably moving tube 180 comes into contact with the formed foam 170 and destroys it, depending on how long the tube 180 was chosen to be. In other words the total length L_{T} of tube 180 determines the height of the foam H_{F}, whereby the distance ΔL between the second end 182 of the tube 180 and the liquid level 155 (i.e. the minimum distance between the second end 182 of the tube 180 and the liquid level 155, here: measured in the centre) defines the height of the foam H_{F}: ΔL=H_{F}. The chaotic movements of tube 180 and the fluid escaping under pressure therefore destroy the foam 170 on contact and thereby control the foam height H_{F} accordingly.

It can be clearly seen in FIG. 3a that in the foam 170, the middle section of the foam layer has been destroyed by the chaotic movement of the tube 180 and by the pressurized fluid exiting from the tube 180, and so the foam layer begins to collapse and reduce significantly. The foam height H_{F} can be kept low in a controlled manner throughout the cultivation method taking place in the bioreactor 100, so that the foam forming method is not affected by the foam 170. An after-formation and uncontrolled increase of the foam 170 or even an over-foaming and escape of the foam 170 from the bioreactor 100 is thus prevented.

Particularly advantageously, foam destruction/control can be carried out when F_{I}/F_{G}<10 or F_{I}/F_{G}<5 or F_{I}/F_{G}<4 or F_{I}/F_{G}<3 or F_{I}/F_{G}<2 applies, in particular if F_{I}/F_{G}<1, where F_{I} is the momentum force according to formula (1) and F_{G} is the weight force according to formula (2) as already explained.

FIG. 3b is identical to the embodiment according to FIG. 3a, however, the single tube 180 is installed in the side wall 140b, wherein the rigid fluid line 185, to which the tube 180 is attached, extends through the side wall 140b. The rigid fluid line can be selected such that the tube 180 is located approximately centrally in the reactor 100 in the rest position and is arranged on the centre axis Z₁ of the bioreactor 100. In particular, in this example case, the attachment point 183 of the tube 180 on the fluid line 185 and the tube 180 in the rest position are located on the centre axis Z₁ of the reactor 100.

In FIG. 3c another embodiment of the present invention is exemplarily illustrated. FIG. 3c shows a reactor 200 which is shown in a schematic simplified sectional view. The reactor 200 may be any type of reactor for any kind of foam-generating method; it may be a chemical reactor or bioreactor including fermenter or another type of reactor. The reactor 200 is in the form of a cylindrical tube with a reactor diameter D_{STR} and an overall height Hₜₒₜₐₗ having a head portion 210 and a bottom part 220 and a central axis Z₂. The reactor 200 may be made of stainless steel or another suitable material. The reactor 200 shown may be used for large-scale production.

The reactor 200 already comprises a liquid medium 250 containing the reactants to be reacted or biological units to be cultivated. A stirrer 260 with a distance h (stirrer bottom clearance) from the bottom 220 of the reactor 200 is provided. The liquid medium 250 is located in the reactor 200 up to a maximum height H, which is the fill level or liquid level 255 of the reactor 200. The liquid medium 250 is not further limited and depends on the foam-generating method taking place in the reactor 200.

In the reactor 200 shown, the foam-generating method is already taking place, so that foam 270 is produced, which accumulates on the liquid medium 250. Foam destruction and/or control is already taking place in the reactor 200 shown. For this purpose, two tubes 280.1 and 280.2 installed in the head portion 210 of the reactor 200, but not in the sidewalls and are each in chaotic motion, symbolised by the double arrows M1 and M2. The first end 281.1 of the first tube 280.1 is attached to a rigid fluid line 285.1 via the attachment point 283.1 and the first end 281.2 of the second tube 280.2 is attached to a rigid fluid line 285.2 via the attachment point 283.2. The tubes 280.1 and 280.2 are both the same length in the example shown. Other embodiments are possible.

The second ends 282.1 and 282.2 of the tubes 280.1 and 280.2 are not fixed and can each move freely. At rest, i.e. when no compressed fluid is flowing through tubes 280.1 and 280.2, tubes 280.1 and 280.2 hang down loosely and each has a total length L_{T}.

The fluid lines 285.1 and 285.2 are connected to a fluid connection (not shown). The compressed fluid flowing through tubes 280.1 and 280.2 is an inert gas such as nitrogen in the example shown. However, another fluid could also be used.

The tubes 280.1 and 280.2 are located in the liquid-free space in the upper part or head portion 210 of the reactor 200, with the two attachment points 283.1 and 283.2 on the rigid fluid lines 285.1 and 285.2 arranged at a distance D_{T} from each other. The distance D_{T} is expediently chosen to be equal or greater than the sum of both lengths of the two tubes 280.1 and 280.2. This way the tubes 280.1 and 280.2 do not touch each other during their movement. The distance of each tube 280.1 and 280.2 to the side walls 240a, 240b, respectively (not shown) is also expediently chosen so that the tubes 280.1 and 280.2 do not come into contact with them.

Therefore, the tubes 280.1 and 280.2 are each spaced apart in the head portion 210 and, in particular, are installed so that the tubes 280.1 and 280.2 do not interfere with each other in their chaotic movement and do not contact the side walls 240a, 240b.

The tubes 280.1 and 280.2 are made of flexible material; examples of this have already been given. The lengths of both tubes 280.1 and 280.2 are selected in such a way that they can each come into contact with the foam 270 during the process and can also come into contact with the liquid level 255 of the liquid medium 250 and can even be temporarily immersed in the liquid medium 250 while foam reduction and/or foam control is being performed.

According to an embodiment, in particular with regard to an economical mode of operation, the inner diameter as well as the wall thickness and thus the weight and flexibility of the tubes 280.1 and 280.2 are selected in such a way that the lowest possible fluid flow rate results for a chaotic movement pattern of the tubes 280.1 and 280.2.

In FIG. 3c the foam destruction and/or control has already started, the tubes 280.1 and 280.2 each move in a chaotic movement pattern. It can be clearly seen in FIG. 3c that the foam 270 has been destroyed by the chaotic movement of the tubes 280.1 and 280.2 and by the pressurised fluid exiting the tubes 280.1 and 280.2, and thus the foam layer begins to degrade and reduce significantly. The foam level can be kept low in a controlled manner throughout the foam-generating method taking place in the reactor 200, so that the foam-generating method is not affected by the foam 270. A post-forming and uncontrolled increase of the foam 270 or even an over-foaming and escape of the foam 270 from the reactor 200 is thus prevented.

Particularly advantageously, the foam destruction/control in FIG. 3c can be carried out if, as already explained, F_{I}/F_{G}<10 or F_{I}/F_{G}<5 or F_{I}/F_{G}<4 or F_{I}/F_{G}<3 or F_{I}/F_{G}<2, in particular if F_{I}/F_{G}<1, where F_{I} is the momentum force according to formula (1) and Fe is the weight force according to formula (2).

In FIGS. 3d to 3f other embodiments of the present invention are exemplarily illustrated. FIGS. 3d to 3f show reactors in schematic simplified sectional views. The reactors 200 may be any type of reactors for any kind of foam-producing method such as chemical reactors or bioreactors including fermenters or another type of reactors. To avoid repetition, only the differences from FIG. 3c are explained here.

In contrast to FIG. 3c, in FIG. 3d the stirrer 360 is driven from above by a stirring rod 365 via a motor (not shown). In the exemplary embodiment shown, the two tubes 280.1 and 280.2 are selected to be of equal length and are installed in the head portion 210, but not in the side walls 240a and 240b. That is the tubes 280.1 and 280.2 are attached to rigid fluid lines 285.1 and 285.2, which extend from the cover of the reactor 200 into the reactor 200. The two tubes 280.1 and 280.2 are spaced apart from each other in the head portion 210. In addition, the length of the both tubes 280.1 and 280.2 is chosen shorter than in FIG. 3c and selected such that the two tubes 280.1 and 280.2 do not come into contact with each other and with the stirring rod 365 and do not touch the side walls 240a and 240b. In this way, the tubes 280.1 and 280.2 are not disturbed in their chaotic movement, so that suitable foam destruction and/or control can take place. In the embodiment shown the tubes 280.1 and 280.2 also do not come into contact with the liquid level 255 of the liquid medium 250. Other variations are also possible.

In FIG. 3e the stirrer 260 is driven from below as shown in FIG. 3c. Furthermore, the first ends 281.1 and 281.2 of the two tubes 280.1 and 280.2 are attached to the rigid fluid lines 285.1 and 285.2 in or nearby the side walls 240a and 240b, respectively. In the exemplary embodiment shown, the two attachment points 283.1 and 283.2 of the two tubes 280.1 and 280.2 are at the same height and at a distance from each other approximately equal to the diameter D_{STR} of the reactor 200. Here, the two tubes 280.1 and 280.2 are of equal length, but do not come into contact with the liquid level 255 of the liquid medium 250. Other variations are also possible. It is understood that the length of the two tubes 280.1 and 280.2 is then chosen in such a way that they do not interfere with each other, but that suitable foam destruction and/or control can still take place. Other configurations are also possible.

In FIG. 3f the stirrer 360 is driven from above by a stirring rod 365 via a motor (not shown). In the exemplary embodiment shown, the two tubes 280.1 and 280.2 are also installed in the side walls 240a and 240b as in FIG. 3e. The length of the two tubes 280.1 and 280.2 is selected such that the two tubes 280.1 and 280.2 do not come into contact with each other and with the stirring rod 365. In this way, the tubes 280.1 and 280.2 are not disturbed in their chaotic movement, so that suitable foam destruction and/or control can take place. In the embodiment shown the tubes 280.1 and 280.2 also do not come into contact with the liquid level 255 of the liquid medium 250. Other variations are also possible.

The advantages of the present invention are extremely manifold:

An advantage of the present invention is in particular that neither chemicals, such as antifoaming agents, nor mechanically-electronically operated or electronically driven components, such as motors, are used to destroy and suppress the foam produced in the method. The disadvantages of antifoaming agents, such as the effect and alteration of product properties, the active intervention and alteration of the method as well as the negative effects on product separation and cleaning are avoided. The disadvantages of mechanically-electronically operated or electronically driven components, such as high expenditure in design and manufacture as well as in repair, maintenance and also cleaning of a complex system are completely eliminated. Sealing against the environment, which is regularly necessary with additional complex structures, is superfluous according to the invention.

The flow-mechanical movement of a flexible tube or, if necessary, several flexible tubes enables the destruction or control of the foam by means of a pressurised fluid, whereby chemical additives for foam destruction can be completely dispensed with. This reduces costs, avoids the introduction of chemical additives and associated impurities as well as the associated negative effects and influences on the product during its manufacture. By dispensing with chemical additives for foam destruction/suppression, the method stability can also be further increased, since the time and quantity of the antifoam agent addition varies and is not predictable, but must always be done as required.

The fluid mechanic solution of the present invention also has the advantage that, if an antifoam agent is additionally used, its quantity can be significantly reduced, thereby increasing method reliability.

The invention can be used in any type of reactor and for any type of foam-generating method. According to an embodiment, the invention can be readily incorporated into existing reactors, for example stainless steel stirred tank reactors, in particular chemical reactors, bioreactors including fermenters or any other type of reactor, and used there. Advantageously, in particular, the device according to the invention and the process according to the invention can be readily adapted to the respective reactor geometry and the type of the respective foam-generating methods and is thus not further limited in application. A change in the design of a reactor is not necessary for this.

According to an embodiment, so-called disposable reactors or SUBs are particularly advantageous, as these are only ever used for the cultivation of one culture and thus have pre-installed tubes and connections. The use of one or more tubes for foam destruction and/or control is therefore particularly simplified.

Instead of one tube, several tubes can also be used according to the invention. The number and position of the tubes depends, for example, on the reactor geometry. In a configuration with a stirrer driven from below, a single tube installed centrally in the reactor, for example, can be advantageous. In the case of a top-driven stirrer, it would be useful if, for example, at least two individual tubes were installed at a suitable distance apart, for example a distance corresponding to the diameter of the reactor, i.e. opposite each other in the side wall.

The maximum foam height H_{F(max)}, which is allowed in the reactor, can be determined in a simple way by the length L_{T} of the tube or tubes.

In order to set the fluid flow rate as low as possible for generating the chaotic movement of the tube or tubes, the parameters of the tube, in particular its internal diameter, can be selected as small as possible and a particularly light and flexible material can be used for the tube.

Particularly advantageous, foam destruction/control can be performed when F_{I}/F_{G}<10 or F_{I}/F_{G}<5 or F_{I}/F_{G}<4 or F_{I}/F_{G}<3 or F_{I}/F_{G}<2 applies, especially if F_{I}/F_{G}<1, where F_{I} is the momentum force according to formula (1) and F_{G} is the weight force according to formula (2).

Overall, the invention is a completely novel concept that has not been described in the prior art before.

### BIBLIOGRAPHY

[1] Schubert, J., L. Wan, and A. Lubbert. Foam suppression by bioreactor retrofitting. Proceedings of the 3rd International Conference on Bioreactor and Bioprocess Fluid Dynamics, Cambridge, UK, Vol. 1416, 1993;
[2] Yoshimura et al.: Recent development of a high density mass culture system for the rotifer Brachionus rotundiformis Tschugunoff, Hydrobiologia 358:139-144, 1997;
[3] Ohkawa et al.: Mechanical Foam Control in a Stirred Draft-tube Bioreactor, J. Chem. Tech. Biotechnol. 1984, 34B, 87-96;
[4] JP 2013074889;
[4a] WO 2008/088371 A2;
[5] CN 202543202 U;
[6] EP 2 758 158 B1;
[7] CN 208684951 U;
[8] US 2014/0293734;
[9] Mostafa et al.: Strategies for Improved dCO2 Removal in Large-Scale Fed-Batch Cultures, Biotechnol. Prog. 2003, 19, 45-51;
[10] DE 20 2020 003748 U1;
[11] US 2016/215248 A1; and
[12] WO 2022/029163 A1.

List of abbreviations:

| Symbol | Description | Unit |
|---|---|---|
| A | cross section | m² |
| d₁ | inner diameter (ID) | m |
| d₂ | outer diameter (OD) | m |
| dₛₜᵢᵣᵣₑᵣ | stirrer diameter | m |
| D_{P} | radial tube position | m |
| D_{T} | installation distance between 2 tubes | m |
| D_{STR} | reactor diameter | m |
| F_{I} | momentum force | N |
| F_{G} | gravity force | N |
| g | acceleration due to gravity | m s⁻² |
| G | weight | kg |
| H_{F} | foam height | mm |
| H_{F(max)} | maximum foam height | mm |
| dH_{F} | foam growth rate | mm s⁻¹ |
| H | liquid level | m |
| Hₜₒₜₐₗ | total reactor height | m |
| h | stirrer bottom clearance | m |
| ID | inner diameter of the tube | m |
| L | length | m |
| ΔL (delta L) | tube distance to maximum liquid height | m |
| L_{T} | tube length | m |
| OD | outer diameter of the tube | m |
| π | circle number or Archimedes' constant (pi) | |
| q | fluid flow rate | m³ s⁻¹ |
| ρ (rho) | density | Kg m⁻³ |
| s | stirrer spacing | m |
| T | temperature | ° C. |
| u | gas exit velocity | m s⁻¹ |
| V | volume | m³ |
| w_{G0} | superficial gas velocity | mm s⁻¹ |
| Z₁, Z₂ | central axis through a reactor | |

### EXAMPLES

### Example 1

Quantification of the foam destruction potential was carried out in an aerated stirred tank reactor on a laboratory scale. The filling volume is 30 L. The experiments were carried out at T=37° C. The foam was generated using a poloxamer (Pluronic® F-68). Poloxamers are block polymers of ethylene oxide and propylene oxide. Demineralised water+1 g/L Pluronic was used as the liquid medium in the reactor. A Rushton turbine (Rt) impeller with a stirring speed of 250 rpm was used as the stirrer in the reactor. Compressed air was used as the compressed fluid. The gas flow rate was 0.02 vvm. The unit “vvm” means volume of air per volume of culture medium per minute, whereby this represents the gas flow rate per volume (vvm=gas flow rate: volume), which therefore results in a gas flow rate of 0.60 L/min, which is converted to 1.0×10⁻⁵ m³/s or 0.00001 m³/s.

A tube was selected for foam destruction, which was installed centrally in the headspace of a stirred tank reactor. The material selected in this example was silicone, so that the tube was flexible, had elasticity and was also mechanically stable.

In this example, the inner diameter, the outer diameter (and thus also the wall thickness) and the length of the tube were varied and the minimum required gas flow rate Vm was determined in each case as a function of these tube parameters, at which the tube performed a chaotic movement pattern. The results are shown in FIG. 4 wherein the measured minimum gas flow rate Vm in [m³/s] (y-axis) was plotted for different tube lengths L in [mm] (x-axis) and different tube diameters of a silicone tube. The diameters of the tube are given in the upper right corner of FIG. 4 as follows:
□ 1×2 mm
Δ 1.5×2.5 mm
◯ 3×4 mm×
x 2×5 mm
⋄ 2×6 mm
The square (1×2 mm) means an inner diameter of 1 mm, an outer diameter of 2 mm, which gives a wall thickness of (2−1):2=0.5 mm.
The triangle (1.5×2.5 mm) means an inner diameter of 1.5 mm, an outer diameter of 2.5 mm and thus a wall thickness of (2.5−1.5):2=0.5 mm.
The circle (3×4 mm) means an inner diameter of 3 mm, an outer diameter of 4 mm and thus a wall thickness of (4−3):2=0.5 mm.
The X (2×5 mm) means an inner diameter of 2 mm, an outer diameter of 5 mm and thus a wall thickness of (5−2):2=1.5 mm.
The rhombus (2×6 mm) means an inner diameter of 2 mm, an outer diameter of 6 mm and thus a wall thickness of (6−2):2=2 mm.

FIG. 4 shows the experimentally determined minimum gas flow rates for different tube diameters and lengths. The minimum gas flow rate is the lowest flow rate of the gas measured at which the tube moves chaotically. Although the tube can already start moving at significantly lower gas flow rates, it does not always move chaotically, but can also initially move only linearly or periodically. To achieve chaotic movement, the gas flow rate is then increased.

From FIG. 4 it can be seen that a small diameter tube (square) requires a low minimum gas flow rate Vm at any length of tube in the range 100 to 700 mm for chaotic movement of the tube to occur. A larger diameter of the tube (rhombus or X) in FIG. 4 shows a much higher minimum gas flow rate Vm required for there to be chaotic movement of the tube at any length of the tube in the range 100 to 700 mm.

It can therefore be seen from FIG. 4 that the smaller the internal diameter of the tube, the less gas flow rate is required to chaotically move the tube. The minimum gas flow rate required to create chaotic movement of the tube therefore increases as the tube diameter increases. The inner diameter of the tube and the gas flow rate therefore determine the gas exit velocity and thus the momentum that moves the tube.

Furthermore, it can be seen from FIG. 4 that the required minimum gas flow rate initially decreases with increasing tube length and then increases again. This can be explained by the fact that the inertial forces dominate with short tube lengths and the weight forces dominate with long tube lengths.

### Example 2

The experimental set-up from example 1 was adopted and a silicone tube was used again. Compressed air was used as the compressed fluid.

The tests were carried out to better quantify the influence of inertial forces and weight forces on the different tube lengths or diameters. For this purpose, the force ratio of momentum force F_{I} to weight force F_{G} was investigated. Silicone tubes with different diameters and lengths were used and the minimum gas flow rate required for the tube to move chaotically was determined. The results are shown in FIG. 5. FIG. 5 shows the dimensionless force ratio F_{I}/F_{G} plotted against the tube length in [mm] for different tube diameters. The individual tube diameters are explained as in FIG. 4. In addition, a best-fit curve was drawn over all tests (linear fit).

The following formula (1) applies to the momentum force F_{I}:

$${F}_{I} = ρ * {u}^{2} * A , \qquad ( 1 )$$

wherein
F_{I} momentum force in [N].
ρ (rho) density of the tube in [kg m⁻³]
u gas exit velocity in [m s⁻¹]
A cross section of the tube in [m²]

The following formula (2) applies to the weight force:

$${F}_{G} = ρ * π / 4 * ( {d}_{2}^{2} - {d}_{1}^{2} ) * g \qquad ( 2 )$$

where
F_{G} weight force in [N]
ρ (rho) density of the tube material in [kg m⁻³]
π circle number or Archimedes' constant (pi): 3.14159265 . . .
d₂ outer diameter (OD) of the tube in [m]
d₁ inner diameter (ID) of the tube in [m]
g acceleration due to gravity in [m s⁻²]

The force ratio F_{I}/F_{G} is a dimensionless value.

FIG. 5 shows that for short tube lengths of 100<L<200 mm, significantly greater momentum forces are required, while from a tube length of L>400 mm a nearly constant ratio of about 0.13 is achieved. Therefore, as can be seen from FIG. 5, in order to ensure a particularly good foam destruction function, a force ratio<10, in particular <5 or <4 or <3 or <2 or <1, was determined. According to some embodiments, a force ratio<1 in particular was found to be advantageous.

FIG. 5 therefore also illustrates the mobility of the tube, i.e. the better the mobility of the tube, the better the foam destruction. I.e. the FIG. 5 shows how effective the tube is and works. From a dimensionless force ratio of F_{I}/F_{G}<1, the weight forces dominate. This means that in the case of chaotic motion, the resulting inertial forces dominate over the applied momentum forces.

### Example 3

The experimental set-up from example 1 was adopted and a flexible tube was used. The tube had a length of 150 mm, an inner diameter of 1 mm and an outer diameter of 2 mm. The tube was made of silicone. The tube is located in the center of the reactor. The stirrer is driven from below. The foam growth was optically recorded during the experiment with a reflex camera. The recording started immediately after the tube was put into operation and performed a chaotic movement. FIG. 6a.1 shows the setup of the reactor, FIG. 6b.1 shows the result without active foam control and FIG. 6c.1 shows the result with active foam control according to the present invention.

FIG. 6a.1 shows the picture of the reactor with a filling volume of 30 L, which is constructed of transparent acrylic glass so that the method in the reactor can be followed during the procedure. The stirrer used driven from below, its rotation speed, the composition of the liquid medium, the temperature, the compressed fluid in the present case a gas and the fluid flow rate in the present case a gas flow rate were as already described for example 1.

FIGS. 6b.1 and 6c.1 show an enlarged section of the reactor shown in FIG. 6a.1 (rectangle in FIG. 6a) in the form of photographs. These photos confirm that the experiments were actually carried out.

For better representability of the gray scale of the photos, FIGS. 6a.2, 6b.2 and 6c.2 show contour representations of the photos of FIGS. 6a.1, 6b.1 and 6c.1.

In FIG. 6b.2, which corresponds to the photo of FIG. 6b.1, the stirring rod 165, the liquid level 155, the formed foam 170 and the height of the foam H_{F} can be seen. In FIGS. 6b.1 and 6b.2 there is no foam control, i.e. the foam destruction device of the present invention is not in operation. In FIG. 6c.2, which corresponds to the photo of FIG. 6c.1, active foam control by the foam destruction device of the invention is taking place. In FIGS. 6b.1 and 6b.2 it can be seen that without foam control, the foam (foam height H_{F}) already makes up ⅔ of the section shown and will soon fill it completely. From FIGS. 6c. 1 and 6c.2 it can be seen that with active foam control at the same time during the process as in FIGS. 6b.1 and 6b.2, a significantly reduced foam height H_{F} is obtained. Therefore, the excessively formed foam is destroyed and controlled to a foam height H_{F} that does not interfere with the method in the reactor.

### Example 4

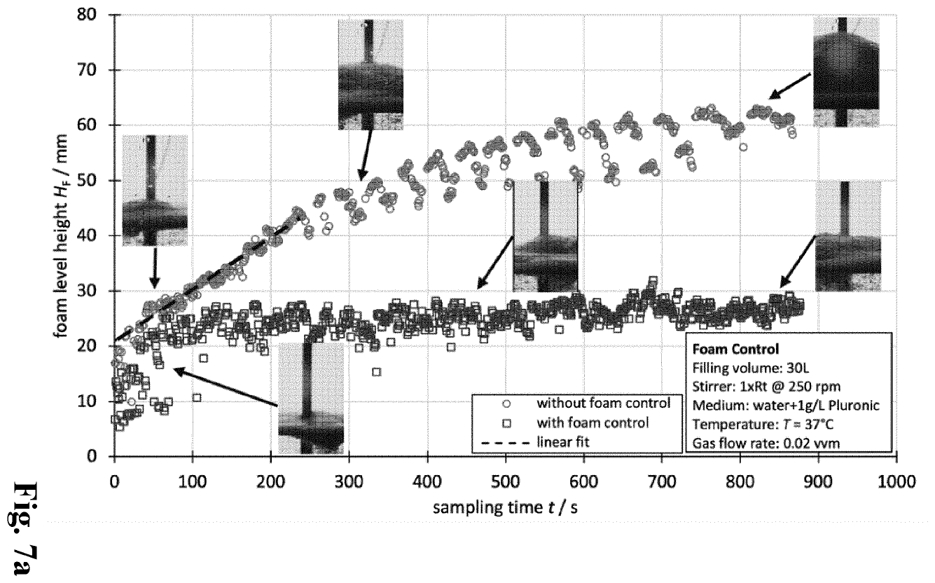

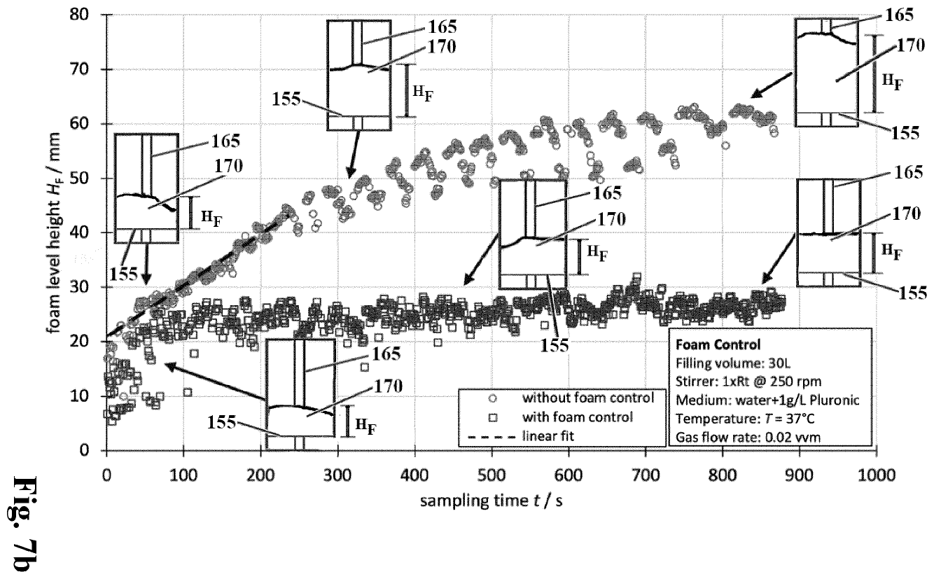

In the reactor of example 3 the experimental set-up from example 1 was adopted and the foam height H_{F} was measured as a function of sampling time. The tube had a length of 150 mm, an inner diameter of 1 mm and an outer diameter of 2 mm. The tube was made of silicone. The results were shown in FIGS. 7a, 7b and 7c. The height of the foam in [mm] was plotted against the sample time in [s]. The upper curve in FIG. 7a (circles) shows the foaming of the method without foam control (without foam destruction device or process of the invention) and the lower curve in FIG. 7a (squares) shows the foaming of the method with active foam control (with foam destruction device or process of the invention). FIG. 7a also shows 3 photos of samples for each curve, showing the respective height of the foam layer at the time shown.

To better represent the grayscale of the photos, in FIG. 7b the upper and lower curves are identical to FIG. 7a, but the photos have been replaced with contour diagrams.

In the contour diagrams of FIG. 7b, which correspond to the respective photos of FIG. 7a, the stirring rod 165, the liquid level 155, the foam 170 and the height of the foam H_{F} are shown.

FIG. 7c shows the measured values with the photos omitted to give a better impression of the curves themselves.

FIGS. 7a, 7b and 7c show in the upper curve (circles) without foam control that the foam grows to a height H_{F} of more than 60 mm while the foam-generating method is running in the reactor. Here, a distinction can be made between two time periods. First, the foam grows almost linearly within the first 200 s from H_{F}=21 mm to H_{F}=40 mm (dashed line). The foam growth rate thus corresponds to dHF=0.09 mm/s, which is very close to a superficial fluid velocity of W_{G0}=0.89 mm/s. This means that the measurement is valid. After that, the foam growth rate flattens out, since not only new foam is generated by the air bubbles, but also “old” foam decays. Since pure poloxamer foam is not particularly stable, equilibrium is reached after approx. 800 s measuring time.

In the lower curve (squares) of FIGS. 7a, 7b and 7c with active foam control according to the present invention, the equilibrium between foam formation and foam destruction is already reached after about 100 s and the foam then remains constant at a level of about 25 mm. Thus, about 25 mm represent the maximum foam height H_{F(max)} in this method. The 25 mm foam height H_{F(max)} corresponds to the installation height of the tube end above the liquid level, i.e. the distance between the second end of the tube and the water level when the tube is at rest and the water level is calm, i.e. ΔL (delta L) in FIGS. 3a, 3b and 3c. This proves that the tube length can indeed be used to keep the foam height H_{F} controlled at the desired height.

### Example 5

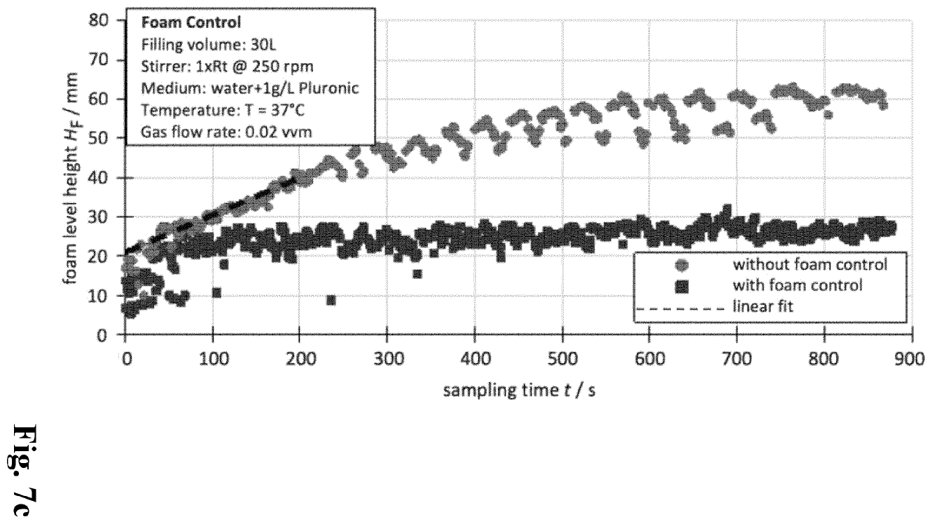

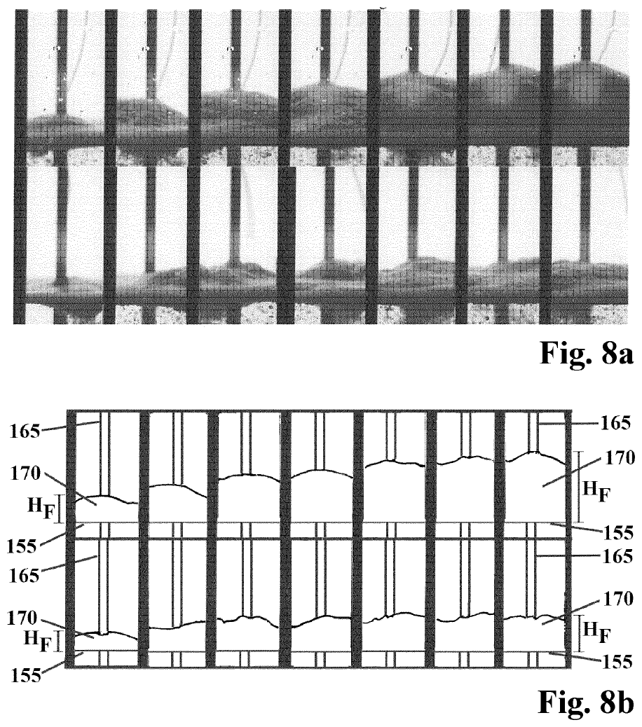

For the reactor of example 3 and the set-up of example 1, the foam height was photographed as a function of sampling time. The tube had a length of 150 mm, an inner diameter of 1 mm and an outer diameter of 2 mm. The tube was made of silicone. The results are shown in FIGS. 8a and 8b.

For better representability of the gray scale of the photos in FIG. 8a, the photos were replaced by contour representations in FIG. 8b. In the contour diagrams of FIG. 8b, which correspond to the respective photos of FIG. 8a, the stirring rod 165, the liquid level 155, the foam 170 and the height of the foam H_{F} are shown.

FIGS. 8a and 8b thus show the foam heights H_{F} in the reactor as chronological sections of the measurements. The upper row of FIG. 8a and FIG. 8b shows foam growth without and the lower row FIG. 8a and FIG. 8b shows foam growth with active foam destruction according to the present invention. There is a time interval of 50s between each image or contour plot.

The chronological progression shown in the upper row of FIG. 8a and FIG. 8b shows the clearly increasing amount of foam 170 in the course of the method when no foam control takes place. In contrast, it can be seen from the lower row of the pictures or contour plots that the foam formation was controlled in a suitable manner by active foam destruction and/or control. The foam height H_{F} remains almost constant at a very low level.

### Example 6

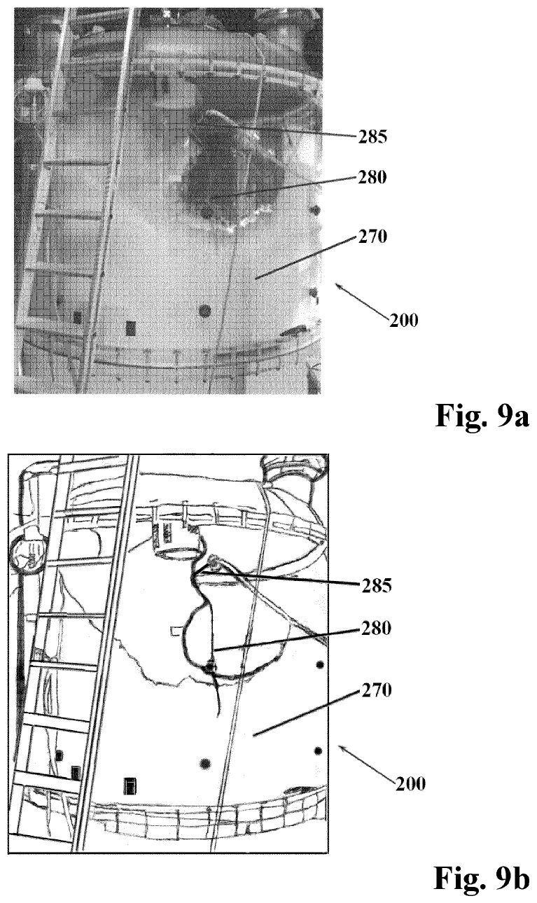

FIG. 9a shows a photo and FIG. 9b shows a contour drawing of the photo of another experiment. This is an aerated industrial scale 12 kL acrylic glass reactor 200. The ladder leaning against it serves as access to the head portion of the reactor 200 and clarifies the dimensions. A silicone tube 280 was connected to the side wall in the headspace of the reactor 200 via a rigid fluid line 285 to a fluid connection, the tube hangs loosely down into the reactor 200 as long as no fluid is flowing through it. In example 6 the fluid is compressed air. The tube 280 had a length of 300 mm, an inner diameter of 5 mm and an outer diameter of 15 mm. The tube 280 was made of silicone. The tube 280 is on the right side in the photo. The photo was taken after the procedure was carried out, and the foam formation was significantly reduced where the tube 280 was installed, i.e. on the right side in the reactor 200, while on the left side in the reactor 200, where the tube 280 did not reach, the foam grew up to the lid.

### Example 7

The experiment of Example 6 was repeated, but instead of compressed air, the liquid medium in the reactor was used as fluid. Also in this case, foam formation was significantly reduced where tube 280 was installed, i.e., on the right side in reactor 200, while on the left side in reactor 200, where tube 280 did not reach, foam filled the reactor up to the lid.

### Example 8

The use of the foam destruction device according to the present invention was investigated in a Single Use Stirred Tank Reactor (SUB). The reactor used was a ThermoFischer HyPerforma™ 2:1 100 L SUB. The inner tank diameter D was 0.44 m with a filling volume V of 100 L. The experiments were carried out at T=20° C. The foam was produced with a poloxamer (Pluronic® F-68). Poloxamers are block polymers of ethylene oxide and propylene oxide. Deionised water+1 g/L Pluronic was used as the liquid medium in the reactor. The stirrer had a stirring speed of 180 rpm. Compressed air was used as the compressed fluid. The gas flow rate was 0.01 vvm. The unit “vvm” means air volume per volume of culture medium per minute, whereby it is the gas flow rate per volume (vvm=gas flow rate: volume) which therefore results in a gas flow rate of 1.0 L/min, the superficial fluid velocity W_{G0} was 0.11 mm/s.

A tube selected for foam destruction was installed in the centre of the head space of the stirred SUB. In this example, silicone was chosen as the material so that the tube is flexible, elastic and also mechanically stable. The outer diameter was 2 mm, the inner diameter was 1 mm and the tube length was 275 mm. The cross-section A of the reactor was 0.15 m². The spacing between the end of the tube and the free surface ΔL, i.e. the minimum distance ΔL between the second end of the tube and the liquid level in the reactor was 18 mm (in the centre), which helps to define the maximum foam height H_{F(max)} of the foam.

The measured foam level height in an aerated single-use stirred tank reactor (SUB) with and without active foam control (with or without foam destruction device or process of the present invention) is summarised in the following table.

TABLE

| Without active foam control | | | | With foam control | |
|---|---|---|---|---|---|
| | | Foam Height | Foam Height | | Foam Height |
| Time | Time | (outer edge*) | (central**) | Time | (outer edge*) |
| [min] | [s] | [mm] | [mm] | [s] | [mm] |
| 0 | 0 | 5 | | 5.01 | 6.06 |
| 1 | 60 | 10 | | 10.01 | 6.78 |
| 2 | 120 | 18 | | 15.02 | 15.10 |
| 3 | 180 | 24 | | 20.02 | 16.29 |
| 4 | 240 | 28 | 60 | 25.03 | 16.76 |
| 5 | 300 | 30 | | 30.03 | 19.14 |
| 6 | 360 | 35 | | 35.04 | 19.73 |
| 7 | 420 | 40 | 100 | 40.04 | 20.92 |
| 8 | 480 | 47 | | 45.05 | 21.63 |
| 9 | 540 | 53 | | 50.05 | 22.70 |
| 10 | 600 | 58 | 165 | 55.06 | 25.68 |
| 11 | 660 | 62 | | 60.06 | 26.03 |
| 12 | 720 | 70 | | 65.07 | 27.70 |
| 13 | 780 | 74 | | 70.07 | 27.70 |
| 14 | 840 | 78 | | 75.08 | 24.60 |
| 15 | 900 | 80 | | 80.08 | 27.82 |
| 16 | 960 | 85 | 220 | 85.09 | 28.65 |
| 17 | 1020 | 92 | | 90.09 | 30.31 |
| 18 | 1080 | 100 | | 95.10 | 30.43 |
| 19 | 1140 | 110 | | 100.1 | 28.05 |
| 20 | 1200 | 120 | 240 | 105.11 | 28.89 |
| 21 | 1260 | 128 | | 110.11 | 30.43 |
| 22 | 1320 | 144 | | 115.12 | 32.45 |
| 23 | 1380 | 150 | | 120.12 | 34.23 |
| 24 | 1440 | 175 | | 125.13 | 33.28 |
| 25 | 1500 | 180 | | 130.13 | 35.90 |
| 26 | 1560 | 190 | | 135.14 | 35.54 |
| | | | | 140.14 | 37.56 |
| | | | | 145.15 | 38.40 |
| | | | | 150.15 | 37.44 |
| | | | | 155.16 | 39.46 |
| | | | | 160.16 | 38.75 |
| | | | | 165.17 | 40.06 |
| | | | | 170.17 | 39.23 |
| | | | | 175.18 | 42.44 |
| | | | | 180.18 | 42.67 |
| | | | | 185.19 | 41.96 |
| | | | | 190.19 | 41.96 |
| | | | | 195.20 | 44.93 |
| | | | | 200.2 | 46.24 |
| | | | | 205.21 | 47.43 |
| | | | | 210.21 | 48.86 |
| | | | | 215.22 | 48.62 |
| | | | | 220.22 | 49.21 |
| | | | | 225.23 | 49.33 |
| | | | | 230.23 | 51.59 |
| | | | | 235.24 | 52.30 |
| | | | | 240.24 | 55.04 |
| | | | | 245.25 | 54.92 |
| | | | | 250.25 | 55.51 |
| | | | | 255.26 | 56.11 |
| | | | | 260.26 | 56.82 |
| | | | | 265.27 | 59.32 |
| | | | | 270.27 | 59.79 |
| | | | | 275.28 | 61.57 |
| | | | | 280.28 | 60.86 |
| | | | | 285.29 | 62.05 |
| | | | | 290.29 | 62.29 |
| | | | | 295.30 | 62.05 |
| | | | | 300.3 | 63.24 |
| | | | | 305.31 | 64.90 |
| | | | | 310.31 | 66.92 |
| | | | | 315.32 | 65.14 |
| | | | | 320.32 | 66.92 |
| | | | | 325.33 | 67.16 |
| | | | | 330.33 | 69.30 |
| | | | | 335.34 | 69.30 |
| | | | | 340.34 | 68.35 |
| | | | | 345.35 | 69.30 |
| | | | | 350.35 | 70.01 |
| | | | | 355.36 | 71.32 |
| | | | | 360.36 | 71.92 |
| | | | | 365.37 | 72.15 |
| | | | | 370.37 | 70.49 |
| | | | | 375.38 | 71.80 |
| | | | | 380.38 | 71.92 |
| | | | | 385.39 | 73.82 |
| | | | | 390.39 | 73.11 |
| | | | | 395.40 | 73.58 |
| | | | | 400.4 | 74.18 |
| | | | | 405.41 | 75.13 |
| | | | | 410.41 | 75.36 |
| | | | | 415.42 | 76.31 |
| | | | | 420.42 | 75.60 |
| | | | | 425.43 | 77.98 |
| | | | | 430.43 | 76.67 |
| | | | | 435.44 | 77.03 |
| | | | | 440.44 | 77.03 |
| | | | | 445.45 | 77.86 |
| | | | | 450.45 | 78.22 |
| | | | | 455.46 | 79.17 |
| | | | | 460.46 | 80.48 |
| | | | | 465.47 | 80.83 |
| | | | | 470.47 | 80.83 |
| | | | | 475.48 | 81.66 |
| | | | | 480.48 | 82.73 |
| | | | | 485.49 | 82.14 |
| | | | | 490.49 | 82.61 |
| | | | | 495.50 | 80.36 |
| | | | | 500.5 | 81.66 |
| | | | | 505.51 | 81.31 |
| | | | | 510.51 | 78.10 |
| | | | | 515.52 | 82.02 |
| | | | | 520.52 | 83.45 |
| | | | | 525.525 | 83.45 |
| | | | | 530.53 | 85.35 |
| | | | | 535.535 | 80.00 |
| | | | | 540.54 | 83.33 |
| | | | | 545.545 | 85.59 |
| | | | | 550.55 | 85.35 |
| | | | | 555.555 | 86.30 |
| | | | | 560.56 | 86.30 |
| | | | | 565.565 | 85.11 |
| | | | | 570.57 | 86.66 |
| | | | | 575.575 | 86.89 |
| | | | | 580.58 | 85.82 |
| | | | | 585.585 | 85.82 |
| | | | | 590.59 | 85.71 |
| | | | | 595.595 | 87.01 |
| | | | | 600.6 | 87.37 |
| | | | | 605.605 | 87.73 |
| | | | | 610.61 | 87.61 |
| | | | | 615.615 | 88.20 |
| | | | | 620.62 | 87.96 |
| | | | | 625.625 | 86.89 |
| | | | | 630.63 | 87.85 |
| | | | | 635.635 | 88.44 |
| | | | | 640.64 | 88.68 |
| | | | | 645.645 | 88.56 |
| | | | | 650.65 | 88.56 |
| | | | | 655.655 | 88.44 |
| | | | | 660.66 | 89.87 |
| | | | | 665.665 | 90.34 |
| | | | | 670.67 | 90.70 |
| | | | | 675.675 | 90.82 |

*Foam height, measured at the inner edge of the reactor, where the outermost edge of the foam is located
**Foam height measured approximately in the centre of the reactor

The measured values in the table above are only given up to about 675 s, but the measurement was continued up to a time of 1600 s. However, the measured values remained essentially constant around 90 mm after a measuring time of 675 s, so that these values are not explicitly listed in the table above.

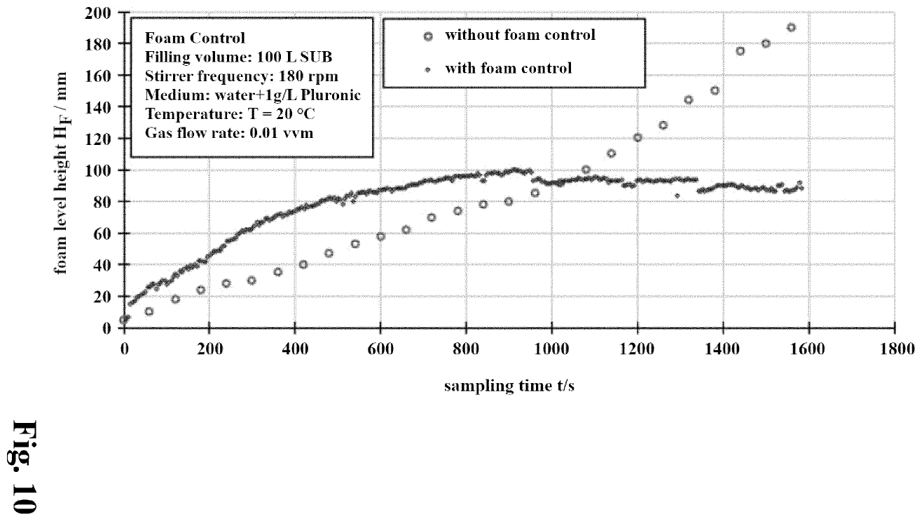

For a better understanding, the measured values of the foam height up to a time of 1600 s are shown in FIG. 10. FIG. 10 therefore shows the foam level height H_{F} measured according to example 8 in [mm] plotted against the measurement time t in [s].

Based on the illustration in FIG. 10, it can be observed that without active foam control, the foam grows linearly over time. This can lead to the reactor over foaming, resulting in the termination of the cultivation. In the case of active foam control according to the present invention, the foam initially grows, then remains constant at a height of about 90 mm. The effect that the foam seems to grow faster with active foam control in the single-use stirred tank reactor (SUB) than without foam control can be explained as follows:

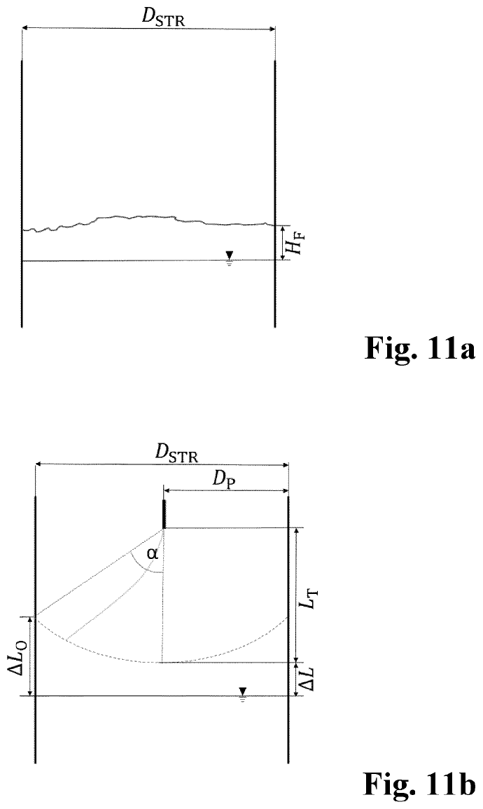

Since, there are no baffles installed in a single-use stirred tank reactor (SUB), the stirrer generates a dominant tangential flow, which causes the gaseous phase to accumulate more in the middle of the reactor. This results in a convex foam layer (see FIG. 11a, H_{F}: foam height, D_{STR} tank diameter). In the case with active foam control according to the present invention, the foam is destroyed by the centrally installed tube. The tube sweeps over the foam in such a way that a concave foam layer is formed (see FIG. 11b, D_{P}: distance between tube and side wall of the reactor; D_{STR}: tank diameter; ΔLₒ: theoretical outer foam height; L_{T}: tube length; ΔL: distance between the second end of the tube and the liquid level in the reactor; alpha: theoretical deflection angle of the tube). At the beginning of the experiment, the distance between the end of the tube and the foam is too large, so that the air impulse does not completely destroy the foam, but instead displaces it radially. The theoretical outer foam height

$$Δ {L}_{o} = Δ L + {L}_{T} ( 1 - sin ( {cos}^{- 1} ( \frac{{D}_{STR}}{2 \cdot {L}_{T}} ) ) )$$

can be geometrically described based on the tube length L_{T}, the tank diameter D_{STR} and the spacing between the tube end and the surface are ΔL (see FIG. 11b). As a result, more foam accumulates at the edge of the water surface and rises faster than without foam control. However, the fact that the foam grows faster at the beginning of the experiment than without foam control is not a problem, because what is decisive is that the foam does not grow beyond a critical height.

In example 8, the minimum distance ΔL between the second end of the tube and the liquid level in the reactor was specified as 18 mm (in the centre). Therefore, the distance of 18 mm refers only to the centre of the surface, i.e. the shortest or minimum distance between the second end of the tube and the liquid level is measured. However, based on the above equation, the distance from the end of the tube to the liquid level at the edge is 128 mm, which is well above the measured maximum foam height of about 90 mm (at the edge). This therefore confirms why the foam at the measuring point (at the edge) initially grows faster with active foam control than without foam control.

Based on FIG. 10 it can be concluded that the described foam destruction device can be also applied to single use aerated stirred tank reactors.

### LIST OF REFERENCE SIGNS

10, 180, 280.1, 280.2 tube
20, 181, 281.1, 281.2 first end of the tube
30, 182, 282.1, 282.2 second end of the tube
40 middle section of the tube
100 bioreactor
110, 210 upper part, head portion
120, 220 lower part, bottom
132, 134 baffles
135 gas bubbles
140a, 140b, 240a, 240b side wall
150, 250 liquid medium
155, 255 liquid level
160, 260, 360 stirrer
165, 265, 365 stirrer rod
167 stirrer motor
170, 270 foam
183, 283.1, 283.2 attachment point
185, 285.1, 285.2 rigid fluid line
200 reactor

### Legends to the Figures

### FIG. 1a:

OD outer diameter of the tube
ID inner diameter of the tube
L_{T} total length L_{T} of the tube

### FIG. 1b:

OD outer diameter of the tube
ID inner diameter of the tube

### FIGS. 2a to 2c:

A linear movement
B periodic or periodically similar movement
C chaotic movement

### FIGS. 3a and 3b:

D_{STR} a reactor diameter
D_{P} distance from the side wall
M chaotic motion of the flexible tube
dₛₜᵢᵣᵣₑᵣ diameter of the stirrer
Z₁ central axis of the reactor
h distance (stirrer bottom clearance)
S distance between two impellers mounted on a stirrer rod
H height of the liquid in the reactor
ΔL (delta L) minimum distance between the second end of the tube and the liquid level
L_{T} total length L_{T} of the tube
Hₜₒₜₐₗ total height of the reactor

### FIGS. 3c and 3e:

D_{STR} a reactor diameter
D_{T} distance of the rigid fluid lines
M1 chaotic motion of a first tube
M2 chaotic motion of a second tube
Z₂ central axis of the reactor
h distance (stirrer bottom clearance)
H height of the liquid in the reactor
L_{T} total length L_{T} of the tube
Hₜₒₜₐₗ total height of the reactor

### FIGS. 3d and 3f:

D_{STR} a reactor diameter
M1 chaotic motion of a first tube
M2 chaotic motion of a second tube
Z₂ central axis of the reactor
h distance (stirrer bottom clearance)
S distance between two impellers mounted on a stirrer rod
H height of the liquid in the reactor
ΔL (delta L) minimum distance between the second end of the tube and the liquid level
Hₜₒₜₐₗ total height of the reactor

### FIG. 4:

### Symbol Inner Diameter of the Tube×Outer Diameter of the Tube:

□ 1×2 mm
Δ 1.5×2.5 mm
◯ 3×4 mm×
x 2×5 mm
⋄ 2×6 mm

### FIG. 5:

### Symbol Inner Diameter of the Tube×Outer Diameter of the Tube:

□ 1×2 mm
Δ 1.5×2.5 mm
◯ 3×4 mm×
x 2×5 mm
⋄ 2×6 mm
[symbol] linear fit

### FIGS. 6a.1 and 6a.2:

Conditions in the Reactor with Active Foam Control/No Foam Control:
Filling volume: 30 L
Stirrer: 1×Rt @ 250 rpm
Medium: water+1 g/L Pluronic
Temperature: T=37° C.
Gas flow rate: 0.02 vvm

### FIGS. 7a and 7b:

### Foam Control:

Filling volume: 30 L
Stirrer: 1×Rt @ 250 rpm
Medium: water+1 g/L Pluronic
Temperature: T=37° C.
Gas flow rate: 0.02 vvm
∘ without foam control
□ with foam control
[symbol] linear fit

### FIG. 7c:

### Foam Control:

Filling volume: 30 L
Stirrer: 1×Rt @ 250 rpm
Medium: water+1 g/L Pluronic
Temperature: T=37° C.
Gas flow rate: 0.02 vvm
● without foam control
▪ with foam control
[symbol] linear fit

### FIG. 8b:

H_{F} foam height

### FIG. 10:

### Foam Control:

Filling volume: 100 L SUB
Stirrer frequency: 180 rpm
Medium: water+1 g/L Pluronic
Temperature: T=20° C.
Gas flow rate: 0.01 vvm
∘ without foam control
* with foam control

### FIG. 11a:

D_{STR} a reactor diameter
H_{F} foam height

### FIG. 11b:

D_{STR} a reactor diameter
D_{P} distance from the side wall
L_{T} total length L_{T} of the tube
ΔL (delta L) minimum distance between the second end of the tube and the liquid level
ΔLₒ (delta Lₒ) theoretical outer foam height
alpha theoretical deflection angle of the tube

1. A foam destruction device for destroying foam (170, 270) and/or controlling the foam height Hp in a reactor (100, 200) in which a foam-generating method is carried out, comprising:
a reactor (100, 200), which is to be filled or is filled with a liquid medium (150, 250) having a liquid level (155, 255);
a flexible tube (10, 180, 280.1, 280.2) having a first end (20, 181, 281.1, 281.2), a second end (30, 182, 282.1, 282.2), a selected length L_{T} and a selected inner and outer diameter;
a rigid fluid line (185, 285.1, 285.2) located above the liquid level (155, 255) of the liquid medium (150, 250) to be filled into the reactor (100, 200) or with which the reactor (100, 200) is filled;
a fluid connection that is connected to the rigid fluid line (185, 285.1, 285.2);
whereby
the first end (20, 181, 281.1, 281.2) of the tube (10, 180, 280.1, 280.2) is attached to the fluid line (185, 285.1, 285.2) to allow compressed fluid to pass through the tube (10, 180, 280.1, 280.2);
the second end (30, 182, 282.1, 282.2) of the tube (10, 180, 280.1, 280.2) is not fixed but is free to move so as to be able to create a chaotic pattern of movement of the second end (30, 182, 282.1, 282.2) of the tube (10, 180, 280.1, 280.2) by the compressed fluid flowing through the tube (10, 180, 280.1, 280.2); and
the length L_{T} of the tube (10, 180, 280.1, 280.2) is chosen such that the second end (30, 182, 282.1, 282.2) of the tube (10, 180, 280.1, 280.2) comes into contact with the liquid level (155, 255) of the liquid medium (150, 250) to be filled into the reactor (100, 200) or with which the reactor (100, 200) is filled or does not come into contact with the liquid level (155, 255) of the liquid medium (150, 250) to be filled into the reactor (100, 200) or with which the reactor (100, 200) is filled.

2. The foam destruction device according to claim 1,
characterized in that
the reactor (100, 200) is selected from a bioreactor (100, 200) or chemical reactor, which is designed for multiple use or single use, in particular a single use bioreactor (SUB).

3. The foam destruction device according to claim 1,
characterized in that
the attachment point (183) of the first end (20, 181) of the tube (10, 180) to the fluid line (185) is located on the central axis (Z₁) of the reactor (100) in the liquid-free upper part (110) of the reactor (100).

4. The foam destruction device according to claim 1,
characterized in that
at least 2 tubes (280.1, 280.2) are provided in the reactor (200) and are attached with their respective first end (281.1, 281.2) to a respective rigid fluid line (285.1, 285.2).

5. The foam destruction device according to claim 1,
characterized in that
the reactor (100, 200) further comprises a stirrer (160, 260, 360) having a stirrer rod (165, 265, 365), a head portion (110, 210) and side walls (140a, 240a, 140b, 240b), whereby
a stirrer (160) is driven from below, a single tube (10, 180) is provided, in particular installed centrally, offset from the centre or installed in a sidewall (140b) of the reactor (100); or
a stirrer (260) is driven from below, at least two tubes (280.1, 280.2) are installed in the head portion (210) but not the side walls (240a, 240b) of the reactor (200), in particular the at least two tubes (280.1, 280.2) are spaced apart from each other in the head portion (210) and the lengths L_{T} of the at least two tubes (280.1, 280.2) are selected in such a manner that the tubes (280.1, 280.2) do not contact each other during their chaotic movement and do not contact the side walls (240a, 240b); or
a stirrer (360) is driven from below, at least 2 tubes (280.1, 280.2) are installed in the side walls (240a, 240b), in particular opposite one another in the reactor (200) whereby the lengths L_{T} of the at least two tubes (280.1, 280.2) are selected in such a manner that the tubes (280.1, 280.2) do not contact each other during their chaotic movement; or
a stirrer (360) is driven from above, at least 2 tubes (280.1, 280.2) are installed in the side walls (240a, 240b), in particular opposite one another in the reactor (200) whereby the lengths L_{T} of the at least two tubes (280.1, 280.2) are selected in such a manner that the tubes (280.1, 280.2) do not contact each other during their chaotic movement and do not contact the stirrer rod (365); or
a stirrer (360) is driven from above, at least 2 tubes (280.1, 280.2) are installed in the head portion (210) but not the side walls (240a, 240b) of the reactor (200), in particular the at least two tubes (280.1, 280.2) are spaced apart from each other in the head portion (210) and the lengths L_{T} of the at least two tubes (280.1, 280.2) are selected in such a manner that the tubes (280.1, 280.2) do not contact each other during their chaotic movement, do not contact the stirrer rod (365) and the side walls (240a, 240b).

6. The foam destruction device according to claim 1,
characterized in that
at least one, two, three or more of the following features are present in the foam destruction device:
the reactor (100, 200) is designed for laboratory scale or industrial scale;
the compressed fluid is selected from a gas of the group consisting of air, oxygen, or an inert gas, in particular nitrogen, or noble gas;
the compressed fluid is selected from a liquid of the group consisting of water, organic solvent(s), the liquid medium (150, 250) used in the reactor (100, 200) or mixtures thereof;
the material of the tube (10, 180, 280.1, 280.2) is selected from the group consisting of silicone, polyvinyl chloride (PVC), rubber, in particular natural rubber, synthetic rubber, in particular vulcanised rubber (rubber), acrylonitrile butadiene rubber (NBR), acrylonitrile butadiene rubber (NBR) with ethylene propylene diene rubber (EPDM), chlorobutadiene rubber (CR neoprene), Teflon, polyether ether ketone (PEEK), polyethylene (PE) and/or polyamide;
for each tube (10, 180, 280.1, 280.2) in the reactor (100, 200) a rigid fluid line (185, 285.1, 285.2) is provided;
the length L_{T} of the tube (10, 180, 280.1, 280.2) being chosen such that the second end (30, 182, 282.1, 282.2) of the tube (10, 180, 280.1, 280.2) does not come into contact with the liquid medium (150, 250) in the reactor (100, 200) but can come into contact with the foam (170, 270) formed on the liquid medium (150, 250);
if the second end (30, 182, 282.1, 282.2) of the tube (10, 180, 280.1, 280.2) does not come into contact with the liquid medium (150, 250) in the reactor (100, 200), the selected length L_{T} of the tube (10, 180, 280.1, 280.2) determines the minimum distance ΔL between the second end (30, 182, 282.1, 282.2) of the tube (10, 180, 280.1, 280.2) and the liquid level (155, 255) in the reactor (100, 200) and thus the maximum foam height H_{F(max)} in the foam-generating method;
when 2 tubes (280.1, 280.2) are provided, the two attachment points (283.1, 283.2) of the two tubes (280.1, 280.2) to the two rigid fluid lines (285.1, 285.2) are provided at the same height and at a distance from each other which is equal to or greater than the sum of the lengths of the two tubes (280.1, 280.2), in particular corresponding to the diameter of the reactor (100, 200);
if more than 2 tubes are provided, they are distributed in the liquid-free upper part of the reactor in such a way that they do not contact each other when the chaotic movement pattern is executed.

7. The foam destruction device according to claim 1,
characterized in that
a force ratio of the momentum force F_{I} to the weight force F_{G} is chosen so that the following formula (3) applies:
$${F}_{I} / {F}_{G} < 10 or \qquad ( 3 )$$
$${F}_{I} / {F}_{G} < 5 or {F}_{I} / {F}_{G} < 4 or {F}_{I} / {F}_{G} < 3 or {F}_{I} / {F}_{G} < 2 ,$$
$$especially {F}_{I} / {F}_{G} < 1 ,$$
wherein the following formula (1) applies to the momentum force:
$${F}_{I} = ρ * {u}^{2} * A , \qquad ( 1 )$$
wherein
F_{I} momentum force in [N]
ρ (rho) density of the tube material in [kg m⁻³]
u gas exit velocity in [m s⁻¹]
A cross section of the tube in [m²]
and the following formula (2) applies to the weight force:
$${F}_{G} = ρ * π / 4 * ( {d}_{2}^{2} - {d}_{1}^{2} ) * g \qquad ( 2 )$$
wherein
F_{G} weight force in [N]
ρ (rho) density of the tube material in [kg m⁻³]
π circle number or Archimedes' constant (pi)
d₂ outer diameter (OD) of the tube in [m]
d₁ inner diameter (ID) of the tube in [m]
g acceleration due to gravity in [m s⁻²].

8. A process for destroying foam (170, 270) and/or controlling the foam height H_{F} in a reactor (100, 200) in which a foam-generating method is to be carried out, comprising the steps of:
providing a reactor (100, 200) in which a method is to be carried out in which foaming occurs comprising a liquid medium (150, 250) having a liquid level (155, 255);
providing a flexible tube (10, 180, 280.1, 280.2) having a first end (20, 181, 281.1, 281.2), a second end (30, 182, 282.1, 282.2), a selected length L_{T} and a selected inner and outer diameter;
providing a rigid fluid line (185, 285.1, 285.2) that is placed above the liquid level (155, 255) of the liquid medium (150, 250) in the reactor (100, 200);
providing a fluid connection that is connected to the rigid fluid line (185, 285.1, 285.2);
attaching a first end (20, 181, 281.1, 281.2) of the tube (10, 180, 280.1, 280.2) to the fluid line (185, 285.1, 285.2) connected to the fluid connection, the second end (30, 182, 282.1, 282.2) of the tube (10, 180, 280.1, 280.2) not being fixed and remaining free to move;
allowing the tube (10, 180, 280.1, 280.2) to hang loosely downwards from the point of attachment (183, 283.1, 283.2) with the fluid line (185, 285.1, 285.2) into the reactor (100, 200), the length L_{T} of the tube (10, 180, 280.1, 280.2) being chosen such that the second end (30, 182, 282.1, 282.2) of the tube (10, 180, 280.1, 280.2) comes into contact with the liquid level (155, 255) of the liquid medium (150, 250) in the reactor (100, 200) or does not come into contact with the liquid level (155, 255) of the liquid medium (150, 250);
as soon as foam (170, 270) forms in the foam-generating method, adjusting the fluid flow through the tube (10, 180, 280.1, 280.2) so that the second end (30, 182, 282.1, 282.2) of the tube (10, 180, 280.1, 280.2) performs a chaotic pattern of movement in the reactor (100, 200); and
destroying the formed foam (170, 270) and/or controlling the foam height H_{F} of the formed foam (170, 270) by contact of the tube (10, 180, 280.1, 280.2) with the foam (170, 270) and by the compressed fluid exiting from the tube (10, 180, 280.1, 280.2).

9. The process according to claim 8,
characterized in that
the reactor (100, 200) is selected from a bioreactor (100, 200) or chemical reactor, which is designed for multiple use or single use, in particular a single use bioreactor (SUB).

10. The process according to claim 8,
characterized in that
the attachment point (183) of the first end of the tube (10, 180) to the fluid line (185) is arranged on the central axis (Z₁) of the reactor (100) in the liquid-free upper part (110) of the reactor (100).

11. The process according to claim 8,
characterized in that
at least 2 tubes (280.1, 280.2) are provided in the reactor (200) and are attached with their respective first end (281.1, 281.2) to a respective rigid fluid line (285.1, 285.2).

12. The process according to claim 8,
characterized in that
the reactor (100, 200) further comprises a stirrer (160, 260, 360) having a stirrer rod (165, 265, 365), a head portion (110, 210) and side walls (140a, 240a, 140b, 240b), whereby
a stirrer (160) driven from below is used, a single tube (10, 180) is provided, in particular installed centrally, offset from the centre or installed in a sidewall (140b) of the reactor (100); or
a stirrer (260) driven from below is used, at least two tubes (280.1, 280.2) are installed in the head portion (210) but not the side walls (240a, 240b) of the reactor (200), in particular the at least two tubes (280.1, 280.2) are spaced apart from each other in the head portion (210) and the lengths L_{T} of the at least two tubes (280.1, 280.2) are selected in such a manner that the tubes (280.1, 280.2) do not contact each other during their chaotic movement and do not contact the side walls (240a, 240b); or
a stirrer (360) driven from below is used, at least 2 tubes (280.1, 280.2) are installed in the side walls (240a, 240b), in particular opposite one another in the reactor (200) whereby the lengths L_{T} of the at least two tubes (280.1, 280.2) are selected in such a manner that the tubes (280.1, 280.2) do not contact each other during their chaotic movement; or
a stirrer (360) driven from above is used, at least 2 tubes (280.1, 280.2) are installed in the side walls (240a, 240b), in particular opposite one another in the reactor (200) whereby the lengths L_{T} of the at least two tubes (280.1, 280.2) are selected in such a manner that the tubes (280.1, 280.2) do not contact each other during their chaotic movement and do not contact the stirrer rod (365); or
a stirrer (360) driven from above is used, at least 2 tubes (280.1, 280.2) are installed in the head portion (210) but not the side walls (240a, 240b) of the reactor (200), in particular the at least two tubes (280.1, 280.2) are spaced apart from each other in the head portion (210) and the lengths L_{T} of the at least two tubes (280.1, 280.2) are selected in such a manner that the tubes (280.1, 280.2) do not contact each other during their chaotic movement, do not contact the stirrer rod (365) and the side walls (240a, 240b).

13. The process according to claim 8,
characterized in that
at least one, two, three or more of the following features are selected for the method:
the reactor (100, 200) is designed for laboratory scale or industrial scale;
the compressed fluid is selected from a gas of the group consisting of air, oxygen, or an inert gas, in particular nitrogen or inert gas;
the compressed fluid is selected from a liquid of the group consisting of water, organic solvent(s), the liquid medium (150, 250) used in the reactor (100, 200) or mixtures thereof;
the length L_{T} of the tube (10, 180, 280.1, 280.2) being chosen such that the second end (30, 182, 282.1, 282.2) of the tube (10, 180, 280.1, 280.2) does not come into contact with the liquid medium (150, 250) in the reactor (100, 200) but can come into contact with the foam (170, 270) formed on the liquid medium (150, 250);
if the second end (30, 182, 282.1, 282.2) of the tube (10, 180, 280.1, 280.2) does not come into contact with the liquid medium (150, 250) in the reactor (100, 200), the length L_{T} of the tube (10, 180, 280.1, 280.2) is selected to determine the minimum distance ΔL between the second end (30, 182, 282.1, 282.2) of the tube (10, 180, 280.1, 280.2) and the liquid level (155, 255) in the reactor (100, 200) and thus the maximum foam height H_{F(max)} in the foam-generating method;
the material of the tube (10, 180, 280.1, 280.2) is selected from the group consisting of silicone, polyvinyl chloride (PVC), rubber, in particular natural rubber, synthetic rubber, in particular vulcanised rubber (rubber), acrylonitrile butadiene rubber (NBR), acrylonitrile butadiene rubber (NBR) with ethylene propylene diene rubber (EPDM), chlorobutadiene rubber (CR neoprene), Teflon, polyether ether ketone (PEEK), polyethylene (PE) and/or polyamide;
a rigid fluid line (185, 285.1, 285.2) is provided for each tube (10, 180, 280.1, 280.2) in the reactor (100, 200);
when two tubes (280.1, 280.2) are provided, the two attachment points (283.1, 283.2) of the two tubes (280.1, 280.2) to the two rigid fluid lines (285.1, 285.2) are provided at the same height and at a distance from each other chosen to be equal to or greater than the sum of the lengths of the two tubes (280.1, 280.2), in particular corresponding to the diameter of the reactor (200);
if more than 2 tubes are provided, they are distributed in the liquid-free part of the reactor in such a way that they do not contact each other when the chaotic movement pattern is executed.

14. The process according to claim 8,
characterized in that
a force ratio momentum force F_{I} to weight force F_{G} is chosen so that the following formula (3) applies:
$${F}_{I} / {F}_{G} < 10 or \qquad ( 3 )$$
$${F}_{I} / {F}_{G} < 5 or {F}_{I} / {F}_{G} < 4 or {F}_{I} / {F}_{G} < 3 or {F}_{I} / {F}_{G} < 2 ,$$
$$especially {F}_{I} / {F}_{G} < 1 ,$$
wherein the following formula (1) applies to the momentum force:
$${F}_{I} = ρ * {u}^{2} * A , \qquad ( 1 )$$
whereby
F_{I} momentum force in [N]
ρ (rho) density of the tube in [kg m⁻³]
u gas exit velocity in [m s⁻¹]
A cross section of the tube in [m²]
and the following formula (2) applies to the weight force:
$${F}_{G} = ρ * π / 4 * ( {d}_{2}^{2} - {d}_{1}^{2} ) * g \qquad ( 2 )$$
whereby
F_{G} weight force in [N]
ρ (rho) density of the tube material in [kg m⁻³]
π circle number or Archimedes' constant (pi)
d₂ outer diameter (OD) of the tube in [m]
d₁ inner diameter (ID) of the tube in [m]
g acceleration due to gravity in [m s⁻²].

15. A method for culturing prokaryotic or eukaryotic cells in liquid cell culture in a bioreactor (100, 200), wherein foam is generated during the method, wherein a foam destroying device according to any one of claims 1 to 7 is used in the bioreactor (100, 200) performing a process for destroying foam (170, 270) and/or controlling the foam height Hp in the reactor (100, 200) according to claim 8.

16. A method for producing a recombinant protein, the method comprising the steps of:
step a) culturing prokaryotic or eukaryotic cells expressing a recombinant protein in cell culture in a bioreactor (100, 200), wherein foam is generated during culturing of the cells;
step b) harvesting the recombinant protein;
step c) purifying the recombinant protein;
wherein in step (a) a foam destroying device according to any one of claims 1 to 7 is used in the bioreactor (100, 200) performing a process for destroying foam (170, 270) and/or controlling foam height H_{F} in the reactor (100, 200) according to claim 8.
